(19) United States
(12) Patent Application Publication
(10) Pub. No.: US 2022/0315983 A1
(43) Pub. Date: Oct. 6, 2022

(54) INTEGRATION OF A PROTEIN COLOCALIZATION DEVICE (PCD) ONTO A MICROFLUIDIC DEVICE

(71) Applicant: Palamedrix, Inc., La Jolla, CA (US)

(72) Inventors: Shane Bowen, Encinitas, CA (US); Paul Rothemund, Pasadena, CA (US); Ashwin Gopinath, Palo Alto, CA (US)

(21) Appl. No.: 17/708,786

(22) Filed: Mar. 30, 2022

Related U.S. Application Data
(60) Provisional application No. 63/168,837, filed on Mar. 31, 2021.

(51) Int. Cl. C12Q 1/6804; G01N 33/543; G16B 50/20

(52) U.S. Cl. CPC C12Q 1/6804; G01N 33/54386; G16B 50/20

(57) ABSTRACT

Provided herein are structures and methods for detecting one or more analyte molecules present in a sample. In some embodiments, the one or more analyte molecules are detected using one or more supramolecular structures. In some embodiments, the supramolecular structures are bi-stable, wherein the supramolecular structures shift from an unstable state to a stable state through interaction with one or more analyte molecules from the sample. In some embodiments, the stable state supramolecular structures are configured to provide a signal for analyte molecule detection and quantification.

### CROSS-REFERENCE TO RELATED APPLICATIONS

The present application claims priority to and the benefit of U.S. Provisional Application No. 63/168,837, filed Mar. 31, 2021, and entitled “INTEGRATION OF A PROTEIN COLOCALIZATION DEVICE (PCD) ONTO A MICROFLUIDIC DEVICE”, the disclosure of which is hereby incorporated by reference herein in its entirety for all purposes.

### BACKGROUND

The current state of personalized healthcare is overwhelmingly genome-centric, predominantly focused on quantifying the genes present within an individual. While such an approach has proven to be extremely powerful, it does not provide a clinician with the complete picture of an individual's health. This is because genes are the “blueprints” of an individual and it merely informs the likelihood of developing an ailment. Within an individual these “blueprints” first need to be transcribed into RNA and then translated into various protein molecules, the real “actors” in the cell, in order to have any effect on the health of an individual.

The concentration of proteins, the interaction between the proteins (protein-protein interactions or PPI), as well as the interaction between proteins and small molecules, are intricately linked to the health of different organs, homeostatic regulatory mechanism as well as the interaction of these systems with the external environment. Hence, quantitative information about proteins as well as PPIs is vital to create a complete picture of an individual's health at a given time point as well as to predict any emerging health issues. For instance, the amount of stress experienced by cardiac muscles (e.g., during a heart attack) can be inferred by measuring the concentration of troponin I/II and myosin light chain present within peripheral blood. Similar protein biomarkers have also been identified, validated and are deployed for a wide variety of organ dysfunctions (e.g., liver disease and thyroid disorders), specific cancers (e.g., colorectal or prostate cancer), and infectious diseases (e.g., HIV and Zika). The interaction between these proteins are also essential for drug development and are increasingly becoming a highly sought-after dataset. The ability to detect and quantify proteins and other molecules, within a given sample of bodily fluids, is an integral component of such healthcare development.

### SUMMARY

The present disclosure generally relates to systems, structures and methods for detection and quantification of analyte molecules in a sample.

Provided herein, in some embodiments, is a method for detecting an analyte molecule present in a sample, the method comprising: a) providing a supramolecular structure comprising: i) a core structure comprising a plurality of core molecules, ii) a capture molecule linked to the core structure at a first location, and iii) a detector molecule linked to the core structure at a second location, wherein the supramolecular structure is in an unstable state, such that the detector molecule is configured to be unbound from the core structure through cleavage of a link therebetween at the second location; b) contacting the sample with the supramolecular structure, such that the supramolecular structure shifts from the unstable state to a stable state wherein the detector molecule and the capture molecule are linked together through binding to the analyte molecule, thereby forming a link between the detector molecule and capture molecule; c) providing a trigger to cleave the link between the detector molecule and the core structure at the second location, wherein the detector molecule remains linked to the core structure through the link with the capture molecule; and d) detecting the analyte molecule based on a signal provided by the supramolecular structure that shifted to the stable state.

Provided herein, in some embodiments, is a method for detecting one or more analyte molecules present in a sample, the method comprising: a) providing a plurality of supramolecular structures, each comprising: i) a core structure comprising a plurality of core molecules, ii) a capture molecule linked to the core structure at a first location, and iii) a detector molecule linked to the core structure at a second location, wherein the supramolecular structure is in an unstable state, such that the detector molecule is configured to be unbound from the core structure through cleavage of a link therebetween at the second location; b) contacting the sample with the plurality of supramolecular structures, such that at least one supramolecular structure shifts from the unstable state to a stable state wherein the corresponding detector molecule and capture molecule are linked together through binding to an analyte molecule of the one or more analyte molecules, thereby forming a link between the corresponding detector molecule and capture molecule; c) providing a trigger to cleave the link between each detector molecule and corresponding core structure at the second location of the plurality of supramolecular structures, wherein the detector molecule for the at least one supramolecular structure that shifted to a stable state remains linked to the corresponding core structure through the link with the corresponding capture molecule; and d) detecting a respective analyte molecule of the one or more analyte molecules based on a signal provided by a respective supramolecular structure of the at least one supramolecular structures that shifted to the stable state. In some embodiments, the method further comprises isolating the plurality of supramolecular structures from any detector molecules unbound from any supramolecular structures that did not shift to a stable state.

In some embodiments, any method disclosed herein further comprising quantifying the concentration of the analyte molecule in the sample. In some embodiments, any method disclosed herein further comprising identifying the detected analyte molecule. In some embodiments, any method disclosed herein further comprising detecting the analyte molecule based on the signal when the analyte molecule is present in the sample at a count of a single molecule or higher. In some embodiments, for any method disclosed herein, the sample comprises a complex biological sample and the method provides for single-molecule sensitivity thereby increasing a dynamic range and quantitative capture of a range of molecular concentrations within the complex biological sample. In some embodiments, for any method disclosed herein, the analyte molecule comprises a protein, a peptide, a peptide fragment, a lipid, a DNA, a RNA, an organic molecule, an inorganic molecule, complexes thereof, or any combinations thereof. In some embodiments, for any method disclosed herein, each supramolecular structure is a nanostructure.

In some embodiments, for any method disclosed herein, each core structure is a nanostructure. In some embodiments, for any method disclosed herein, the plurality of core molecules for each core structure are arranged into a pre-defined shape and/or have a prescribed molecular weight. In some embodiments, the pre-defined shape is configured to limit or prevent cross-reactivity with another supramolecular structure. In some embodiments, for any method disclosed herein, the plurality of core molecules for each core structure comprises one or more nucleic acid strands, one or more branched nucleic acids, one or more peptides, one or more small molecules, or combinations thereof. In some embodiments, for any method disclosed herein, each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

In some embodiments, for any method disclosed herein, the trigger comprises a deconstructor molecule, a trigger signal, or combinations thereof. In some embodiments, the deconstructor molecule comprises DNA, RNA, a peptide, a small organic molecule, or combinations thereof. In some embodiments, the trigger signal comprises an optical signal, an electrical signal, or both. In some embodiments, the trigger optical signal comprises a microwave signal, an ultraviolet illumination, a visible illumination, a near infrared illumination, or combinations thereof.

In some embodiments, for any method disclosed herein, the respective analyte molecule is 1) bound to the capture molecule of the respective supramolecular structure through a chemical bond and/or 2) bound to the detector molecule of the respective supramolecular structure through a chemical bond. In some embodiments, for any method disclosed herein, the capture molecule and detector molecule for each supramolecular structure independently comprise a protein, a peptide, an antibody, an aptamer (RNA and DNA), a fluorophore, a darpin, a catalyst, a polymerization initiator, a polymer like PEG, or combinations thereof. In some embodiments, for any method disclosed herein, wherein for each supramolecular structure: a) the capture molecule is linked to the core structure through a capture barcode, wherein the capture barcode comprises a first capture linker, a second capture linker, and a capture bridge disposed between the first and second capture linkers, wherein the first capture linker is bound to a first core linker that is bound to the first location on the core structure, wherein the capture molecule and the second capture linker are linked together through binding to a third capture linker, and b) the detector molecule is linked to the core structure through a detector barcode, wherein the detector barcode comprises a first detector linker, a second detector linker, and a detector bridge disposed between the first and second detector linkers, wherein the first detector linker is bound to a second core linker that is bound to the second location on the core structure, wherein the detector molecule and the second detector linker are linked together through binding to a third detector linker. In some embodiments, the capture bridge and detector bridge independently comprise a polymer core. In some embodiments, the polymer core of the capture bridge and the polymer core of the detector bridge independently comprise a nucleic acid (DNA or RNA) of specific sequence or a polymer like PEG. In some embodiments, the first core linker, second core linker, first capture linker, second capture linker, third capture linker, first detector linker, second detector linker, and third detector linker independently comprise a reactive molecule or DNA sequence domain. In some embodiments, each reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the linkage between the capture barcode and 1) the first core linker, and/or 2) the third capture linker comprises a chemical bond. In some embodiments, the chemical bond comprises a covalent bond. In some embodiments, the linkage between the detector barcode and 1) the second core linker, and/or 2) the third detector linker comprises a chemical bond. In some embodiments, the chemical bond comprises a covalent bond. In some embodiments, the trigger cleaves the linkage between 1) the first detector linker and the second core linker and/or 2) the first capture linker and the first core linker. In some embodiments, for any method disclosed herein, the capture molecule is bound to the third capture linker through a chemical bond and/or the detector molecule is bound to the third detector linker through a chemical bond. In some embodiments, the capture molecule is covalently bonded to the third capture linker and/or the detector molecule is covalently bonded to the third detector linker.

In some embodiments, for any method disclosed herein, each supramolecular structure in the unstable state comprises the respective capture molecule and detector molecule spaced apart at a pre-determined distance, so as to reduce or inhibit the occurrence of cross-reactions between capture and/or detector molecules of a first supramolecular structure and corresponding capture and/or detector molecules of a second supramolecular structure. In some embodiments, for any method disclosed herein, the pre-determined distance is from about 3 nm to about 40 nm.

In some embodiments, for any method disclosed herein, each supramolecular structure further comprises an anchor molecule linked to the core structure. In some embodiments, the anchor molecule is linked to the core structure via an anchor barcode, wherein the anchor barcode comprises a first anchor linker, a second anchor linker, and an anchor bridge disposed between the first and second anchor linkers, wherein the first anchor linker is bound to a third core linker that is bound to a third location on the core structure, wherein the anchor molecule is linked to the second anchor linker. In some embodiments, the anchor molecule comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor bridge comprises a polymer core. In some embodiments, the polymer core of the anchor bridge comprises a nucleic acid (DNA or RNA) of specific sequence or a polymer like PEG. In some embodiments, the third core linker, first anchor linker, second anchor linker, and anchor molecule independently comprise an anchor reactive molecule or DNA sequence domain. In some embodiments, each anchor reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor molecule is linked to the second anchor linker through a chemical bond. In some embodiments, the anchor molecule is covalently bonded to the second anchor linker. In some embodiments, wherein the trigger further cleaves 1) the second anchor linker from the anchor molecule, 2) the first anchor linker from the third core linker, or combinations thereof. In some embodiments, the first and second locations are situated on a first side of the core structure, and the third location is situated on a second side of the core structure.

In some embodiments, for any method disclosed herein, the signal comprises the detector barcode, the capture barcode, or combinations thereof, corresponding to a supramolecular structure that shifted to a stable state. In some embodiments, any method disclosed herein, further comprising separating each detector barcode from a corresponding detector molecule for the at least one supramolecular structure that shifted to a stable state, such that the corresponding signal comprises the respective detector barcode for detection of the analyte molecule bound to the respective capture and detector molecules. In some embodiments, each separated detector barcode provides a DNA signal corresponding to the analyte molecule bound to the respective detector molecule. In some embodiments, the at least one separated detector barcodes are analyzed using genotyping, qPCR, sequencing, or combinations thereof. In some embodiments, a plurality of analyte molecules in the sample are detected simultaneously through multiplexing via one or more supramolecular structures that shifted to a stable state. In some embodiments, for any method disclosed herein, the capture and detector molecules for each supramolecular structure is configured for binding to one or more specific types of analyte molecules.

In some embodiments, for any method comprising using a plurality of supramolecular structures disclosed herein, each core structure of the plurality of supramolecular structures are identical to each other. In some embodiments, each supramolecular structure comprises a prescribed shape, size, molecular weight, or combinations thereof, so as to reduce or eliminate cross-reactions between a plurality of supramolecular structures. In some embodiments, each supramolecular structure comprises a plurality of capture and detector molecules. In some embodiments, each supramolecular structure comprises a prescribed stoichiometry of the capture and detector molecules so as to reduce or eliminate cross-reactions between the plurality of supramolecular structures.

In some embodiments, for any method comprising using a plurality of supramolecular structures disclosed herein, the unstable state for each supramolecular structure further comprises the capture and detector molecules spaced apart at a pre-determined distance so as to reduce or inhibit the occurrence of cross-reactions between capture and/or detector molecules of a first supramolecular structure and a second supramolecular structure. In some embodiments, the pre-determined distance is from about 3 nm and about 40 nm. In some embodiments, the mean distance between any two supramolecular structures is larger than the pre-determined distance between the capture and detector molecules of a respective supramolecular structure. In some embodiments, the plurality of supramolecular structures are attached to one or more widgets, one or more solid supports, one or more polymer matrices, one or more solid substrates, one or more molecular condensates, or combinations thereof. In some embodiments, the mean distance between any two supramolecular structures is larger than the pre-determined distance between the capture and detector molecules of a respective supramolecular structure. In some embodiments, each polymer matrix of the one or more polymer matrices comprises a hydrogel bead. In some embodiments, one or more supramolecular substrates are attached to a hydrogel bead. In some embodiments, each supramolecular structure is co-polymerized with the hydrogel bead through a corresponding anchor molecule linked to the respective core structure of the corresponding supramolecular structure. In some embodiments, the one or more supramolecular structures are embedded within the hydrogel bead. In some embodiments, each hydrogel bead is contacted with a single cell in the sample for intracellular analyte molecule detection at a single cell resolution. In some embodiments, each solid substrate of the one or more solid substrates comprises a microparticle. In some embodiments, one or more supramolecular substrates are attached to a solid surface of the microparticle. In some embodiments, the microparticle comprises a polystyrene particle, silica particle, magnetic particle, or paramagnetic particle. In some embodiments, each solid substrate is contacted with a single cell in the sample for intracellular analyte molecule detection at a single cell resolution. In some embodiments, each solid substrate of the one or more solid substrates comprises a planar substrate. In some embodiments, a plurality of supramolecular structures are disposed on the planar substrate, wherein the planar substrate comprises a plurality of binding sites, wherein each binding site is configured to link with a corresponding supramolecular structure. In some embodiments, the plurality of supramolecular structures are configured to detect the same analyte molecule. In some embodiments, for any method comprising using a planar substrate, further comprising providing a plurality of signaling elements configured to link with the detector molecules of the at least one supramolecular structure that shifted to the stable state. In some embodiments, each signaling element comprises a fluorescent molecule or microbead, a fluorescent polymer, highly charged nanoparticles or polymer. In some embodiments, at least one supramolecular structure of the plurality of supramolecular structures is configured to detect a different analyte molecule from the other supramolecular structures. In some embodiments, for any method comprising using a planar substrate, further comprising barcoding each supramolecular structure so as to identify the location of each supramolecular structure on the planar substrate. In some embodiments, for any method comprising using a planar substrate, further comprising providing a plurality of signaling elements configured to link with the detector molecules of the at least one supramolecular structure that shifted to the stable state. In some embodiments, each signaling element comprises a fluorescent molecule or microbead, a fluorescent polymer, highly charged nanoparticles or polymer.

In some embodiments, for any method disclosed herein, the sample comprises a biological particle or a biomolecule. In some embodiments, for any method disclosed herein, the sample comprises an aqueous solution comprising a protein, a peptide, a fragment of a peptide, a lipid, DNA, RNA, an organic molecule, a viral particle, an exosome, an organelle, or any complexes thereof. In some embodiments, for any method disclosed herein, the sample comprises a tissue biopsy, blood, blood plasma, Urine, Saliva, Tear, Cerebrospinal fluid, extracellular fluid, cultures cells, culture media, discarded tissue, plant matter, a synthetic protein, a bacterial and/or viral sample or fungal tissue, or combinations thereof.

Provided herein, in some embodiments, is a substrate for detecting one or more analyte molecules in a sample, the substrate comprising a plurality of supramolecular structures, each supramolecular structure comprising: a) a core structure comprising a plurality of core molecules, b) a capture molecule linked to the supramolecular core at a first location, and c) a detector molecule linked to the supramolecular core at a second location, wherein the supramolecular structure is in an unstable state, such that the detector molecule is configured to be unbound from the core structure through cleavage of a link therebetween at the second location; wherein each supramolecular structure is configured to shift from the unstable state to a stable state through interaction between the detector molecule, the capture molecule, and a respective analyte molecule of the one or more analyte molecules; wherein, upon interaction with a trigger, a respective supramolecular structure that shifted to the stable state provides a signal for detecting the respective analyte molecule.

In some embodiments, wherein upon interaction with the trigger, each detection molecule linked to a supramolecular structure in the unstable state becomes unbound from said supramolecular structure. In some embodiments, each core structure of the plurality of supramolecular structures is identical to each other. In some embodiments, the mean distance between any two supramolecular structures is larger than the pre-determined distance between the capture and detector molecules of a respective supramolecular structure. In some embodiments, the substrate comprises a solid support, solid substrate, a polymer matrix, or a molecular condensate. In some embodiments, the sample comprises a complex biological sample and the method provides for single-molecule sensitivity thereby increasing a dynamic range and quantitative capture of a range of molecular concentrations within the complex biological sample. In some embodiments, the one or more analyte molecules comprises a protein, a peptide, a peptide fragment, a lipid, a DNA, a RNA, an organic molecule, an inorganic molecule, complexes thereof, or any combinations thereof. In some embodiments, each supramolecular structure is a nanostructure. In some embodiments, each core structure is a nanostructure. In some embodiments, the plurality of core molecules for each core structure are arranged into a pre-defined shape and/or have a prescribed molecular weight. In some embodiments, the pre-defined shape is configured to limit or prevent cross-reactivity with another supramolecular structure. In some embodiments, the plurality of core molecules for each core structure comprises one or more nucleic acid strands, one or more branched nucleic acids, one or more peptides, one or more small molecules, or combinations thereof. In some embodiments, each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof. In some embodiments, the trigger comprises a deconstructor molecule, a trigger signal, or combinations thereof. In some embodiments, the deconstructor molecule comprises DNA, RNA, a peptide, a small organic molecule, or combinations thereof. In some embodiments, the trigger signal comprises an optical signal, an electrical signal, or both. In some embodiments, the trigger optical signal comprises a microwave signal, an ultraviolet illumination, a visible illumination, a near infrared illumination, or combinations thereof. In some embodiments, the respective analyte molecule is 1) bound to the capture molecule through a chemical bond and/or 2) bound to the detector molecule through a chemical bond. In some embodiments, the capture molecule and detector molecule for each supramolecular structure independently comprise a protein, a peptide, an antibody, an aptamer (RNA and DNA), a fluorophore, a darpin, a catalyst, a polymerization initiator, a polymer like PEG, or combinations thereof.

In some embodiments, wherein for each supramolecular structure of the substrate: a) the capture molecule is linked to the core structure through a capture barcode, wherein the capture barcode comprises a first capture linker, a second capture linker, and a capture bridge disposed between the first and second capture linkers, wherein the first capture linker is bound to a first core linker that is bound to the first location on the core structure, wherein the capture molecule and the second capture linker are linked together through binding to a third capture linker, and b) the detector molecule is linked to the core structure through a detector barcode, wherein the detector barcode comprises a first detector linker, a second detector linker, and a detector bridge disposed between the first and second detector linkers, wherein the first detector linker is bound to a second core linker that is bound to the second location on the core structure, wherein the detector molecule and the second detector linker are linked together through binding to a third detector linker. In some embodiments, the capture bridge and detector bridge independently comprise a polymer core. In some embodiments, wherein the polymer core of the capture bridge and the polymer core of the detector bridge independently comprise a nucleic acid (DNA or RNA) of specific sequence or a polymer like PEG. In some embodiments, the first core linker, second core linker, first capture linker, second capture linker, third capture linker, first detector linker, second detector linker, and third detector linker independently comprise a reactive molecule or DNA sequence domain. In some embodiments, each reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the linkage between the capture barcode and 1) the first core linker, and/or 2) the third capture linker comprises a chemical bond. In some embodiments, the chemical bond comprises a covalent bond. In some embodiments, the linkage between the detector barcode and 1) the second core linker, and/or 2) the third detector linker comprises a chemical bond. In some embodiments, the chemical bond comprises a covalent bond. In some embodiments, the trigger cleaves the linkage between 1) the first detector linker and the second core linker and/or 2) the first capture linker and the first core linker. In some embodiments, the capture molecule is bound to the third capture linker through a chemical bond and/or the detector molecule is bound to the third detector linker through a chemical bond. In some embodiments, the capture molecule is covalently bonded to the third capture linker and/or the detector molecule is covalently bonded to the third detector linker. In some embodiments, each supramolecular structure in the unstable state comprises the respective capture molecule and detector molecule spaced apart at a pre-determined distance, so as to reduce or inhibit the occurrence of cross-reactions between capture and/or detector molecules of a first supramolecular structure and corresponding capture and/or detector molecules of a second supramolecular structure. The pre-determined distance is from about 3 nm to about 40 nm.

In some embodiments, each supramolecular structure further comprises an anchor molecule linked to the core structure. In some embodiments, the anchor molecule is linked to the core structure via an anchor barcode, wherein the anchor barcode comprises a first anchor linker, a second anchor linker, and an anchor bridge disposed between the first and second anchor linkers, wherein the first anchor linker is bound to a third core linker that is bound to a third location on the core structure, wherein the anchor molecule is linked to the second anchor linker. In some embodiments, the anchor molecule comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor bridge comprises a polymer core. In some embodiments, the polymer core of the anchor bridge comprises a nucleic acid (DNA or RNA) of specific sequence or a polymer like PEG. In some embodiments, the third core linker, first anchor linker, second anchor linker, and anchor molecule independently comprise an anchor reactive molecule or DNA sequence domain. In some embodiments, each anchor reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor molecule is linked to the second anchor linker through a chemical bond. In some embodiments, the anchor molecule is covalently bonded to the second anchor linker. In some embodiments, the trigger further cleaves 1) the second anchor linker from the anchor molecule, 2) the first anchor linker from the third core linker, or combinations thereof. In some embodiments, the first and second locations are situated on a first side of the core structure, and the third location is situated on a second side of the core structure.

In some embodiments, the signal comprises the detector barcode, the capture barcode, or combinations thereof, corresponding to a supramolecular structure that shifted to a stable state. In some embodiments, each detector barcode from a corresponding detector molecule for the at least one supramolecular structure that shifted to a stable state is configured to be separated from said corresponding detector molecule, such that the corresponding signal comprises the respective detector barcode for detection of the analyte molecule bound to said corresponding detector molecule. In some embodiments, each separated detector barcode provides a DNA signal corresponding to the analyte molecule bound to the respective detector molecule. In some embodiments, the at least one separated detector barcode is configured to be analyzed using genotyping, qPCR, sequencing, or combinations thereof. In some embodiments, one or more supramolecular structures are configured for multiplexing the sample, wherein a plurality of analyte molecules in the sample are detected simultaneously. In some embodiments, the capture and detector molecules for each supramolecular structure is configured for binding to one or more specific types of analyte molecules.

In some embodiments, each core structure of the plurality of supramolecular structures are identical to each other. In some embodiments, each supramolecular structure comprises a prescribed shape, size, molecular weight, or combinations thereof, so as to reduce or eliminate cross-reactions between a plurality of supramolecular structures. In some embodiments, each supramolecular structure comprises a plurality of capture and detector molecules. In some embodiments, each supramolecular structure comprises a prescribed stoichiometry of the capture and detector molecules so as to reduce or eliminate cross-reactions between the plurality of supramolecular structures. In some embodiments, the unstable state for each supramolecular structure further comprises the capture and detector molecules spaced apart at a pre-determined distance so as to reduce or inhibit the occurrence of cross-reactions between capture and/or detector molecules of a first supramolecular structure and a second supramolecular structure. In some embodiments, the pre-determined distance is from about 3 nm and about 40 nm. In some embodiments, the mean distance between any two supramolecular structures is larger than the pre-determined distance between the capture and detector molecules of a respective supramolecular structure.

In some embodiments, each substrate comprises a widget, a solid support, a polymer matrix, a solid substrate, or a molecular condensate. In some embodiments, the mean distance between any two supramolecular structures is larger than the pre-determined distance between the capture and detector molecules of a respective supramolecular structure. In some embodiments, the polymer matrix comprises a hydrogel bead. In some embodiments, one or more supramolecular substrates are attached to the hydrogel bead. In some embodiments, each supramolecular structure is co-polymerized with the hydrogel bead through a corresponding anchor molecule linked to the respective core structure of the corresponding supramolecular structure. In some embodiments, the one or more supramolecular structures are embedded within the hydrogel bead. In some embodiments, each hydrogel bead is configured to be contacted with a single cell in the sample for intracellular analyte molecule detection at a single cell resolution. In some embodiments, the solid substrate comprises a microparticle. In some embodiments, one or more supramolecular substrates are attached to a solid surface of the microparticle. In some embodiments, the microparticle comprises a polystyrene particle, silica particle, magnetic particle, or paramagnetic particle. In some embodiments, each solid substrate is configured to be contacted with a single cell in the sample for intracellular analyte molecule detection at a single cell resolution. In some embodiments, the solid substrate comprises a planar substrate. In some embodiments, a plurality of supramolecular structures are disposed on the planar substrate, wherein the planar substrate comprises a plurality of binding sites, wherein each binding site is configured to link with a corresponding supramolecular structure. In some embodiments, the plurality of supramolecular structures are configured to detect the same analyte molecule. In some embodiments, a plurality of signaling elements are configured to link with the detector molecules of the at least one supramolecular structure that shifted to the stable state. In some embodiments, each signaling element comprises a fluorescent molecule or microbead, a fluorescent polymer, highly charged nanoparticles or polymer. In some embodiments, at least one supramolecular structure of the plurality of supramolecular structures is configured to detect a different analyte molecule from the other supramolecular structures.

In some embodiments, the sample comprises a biological particle or a biomolecule. In some embodiments, the sample comprises an aqueous solution comprising a protein, a peptide, a fragment of a peptide, a lipid, DNA, RNA, an organic molecule, a viral particle, an exosome, an organelle, or any complexes thereof. In some embodiments, the sample comprises a tissue biopsy, blood, blood plasma, Urine, Saliva, Tear, Cerebrospinal fluid, extracellular fluid, cultures cells, culture media, discarded tissue, plant matter, a synthetic protein, a bacterial and/or viral sample or fungal tissue, or combinations thereof.

Provided herein, in some embodiments, is a supramolecular structure for detecting an analyte molecule in a sample, the supramolecular structure comprising: a) a core structure comprising a plurality of core molecules, b) a capture molecule linked to the supramolecular core at a first location, and c) a detector molecule linked to the supramolecular core at a second location, wherein the supramolecular structure is in an unstable state, such that the detector molecule is configured to be unbound from the core structure through cleavage of a link therebetween at the second location; wherein the supramolecular structure is configured to shift from the unstable state to a stable state through interaction between the detector molecule, the capture molecule, and an analyte molecule; wherein, upon interaction with a trigger, the supramolecular structure that shifted to the stable state provides a signal for detecting the analyte molecule.

In some embodiments, the sample comprises a complex biological sample and the method provides for single-molecule sensitivity thereby increasing a dynamic range and quantitative capture of a range of molecular concentrations within the complex biological sample. In some embodiments, the analyte molecule comprises a protein, a peptide, a peptide fragment, a lipid, a DNA, a RNA, an organic molecule, an inorganic molecule, complexes thereof, or any combinations thereof. In some embodiments, the supramolecular structure is a nanostructure. In some embodiments, the core structure is a nanostructure. In some embodiments, the plurality of core molecules for the core structure are arranged into a pre-defined shape and/or have a prescribed molecular weight. In some embodiments, the pre-defined shape is configured to limit or prevent cross-reactivity with another supramolecular structure. In some embodiments, the plurality of core molecules for each core structure comprises one or more nucleic acid strands, one or more branched nucleic acids, one or more peptides, one or more small molecules, or combinations thereof. In some embodiments, the core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof. In some embodiments, the trigger comprises a deconstructor molecule, a trigger signal, or combinations thereof. In some embodiments, the deconstructor molecule comprises DNA, RNA, a peptide, a small organic molecule, or combinations thereof. In some embodiments, the trigger signal comprises an optical signal, an electrical signal, or both. In some embodiments, the trigger optical signal comprises a microwave signal, an ultraviolet illumination, a visible illumination, a near infrared illumination, or combinations thereof. In some embodiments, the analyte molecule is 1) bound to the capture molecule through a chemical bond and/or 2) bound to the detector molecule through a chemical bond. In some embodiments, the capture molecule and detector molecule for each supramolecular structure independently comprise a protein, a peptide, an antibody, an aptamer (RNA and DNA), a fluorophore, a darpin, a catalyst, a polymerization initiator, a polymer like PEG, or combinations thereof.

In some embodiments, where for the supramolecular structure: a) the capture molecule is linked to the core structure through a capture barcode, wherein the capture barcode comprises a first capture linker, a second capture linker, and a capture bridge disposed between the first and second capture linkers, wherein the first capture linker is bound to a first core linker that is bound to the first location on the core structure, wherein the capture molecule and the second capture linker are linked together through binding to a third capture linker, and b) the detector molecule is linked to the core structure through a detector barcode, wherein the detector barcode comprises a first detector linker, a second detector linker, and a detector bridge disposed between the first and second detector linkers, wherein the first detector linker is bound to a second core linker that is bound to the second location on the core structure, wherein the detector molecule and the second detector linker are linked together through binding to a third detector linker. In some embodiments, the capture bridge and detector bridge independently comprise a polymer core. In some embodiments, the polymer core of the capture bridge and the polymer core of the detector bridge independently comprise a nucleic acid (DNA or RNA) of specific sequence or a polymer like PEG. In some embodiments, the first core linker, second core linker, first capture linker, second capture linker, third capture linker, first detector linker, second detector linker, and third detector linker independently comprise a reactive molecule or DNA sequence domain. In some embodiments, each reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the linkage between the capture barcode and 1) the first core linker, and/or 2) the third capture linker comprises a chemical bond. In some embodiments, the chemical bond comprises a covalent bond. In some embodiments, the linkage between the detector barcode and 1) the second core linker, and/or 2) the third detector linker comprises a chemical bond. In some embodiments, the chemical bond comprises a covalent bond. In some embodiments, the trigger cleaves the linkage between 1) the first detector linker and the second core linker and/or 2) the first capture linker and the first core linker. In some embodiments, the capture molecule is bound to the third capture linker through a chemical bond and/or the detector molecule is bound to the third detector linker through a chemical bond. In some embodiments, the capture molecule is covalently bonded to the third capture linker and/or the detector molecule is covalently bonded to the third detector linker. In some embodiments, the supramolecular structure in the unstable state comprises the respective capture molecule and detector molecule spaced apart at a pre-determined distance, so as to reduce or inhibit the occurrence of cross-reactions between capture and/or detector molecules of the supramolecular structure with corresponding capture and/or detector molecules of another supramolecular structure. In some embodiments, the pre-determined distance is from about 3 nm to about 40 nm.

In some embodiments, the supramolecular structure further comprises an anchor molecule linked to the core structure. In some embodiments, the anchor molecule is linked to the core structure via an anchor barcode, wherein the anchor barcode comprises a first anchor linker, a second anchor linker, and an anchor bridge disposed between the first and second anchor linkers, wherein the first anchor linker is bound to a third core linker that is bound to a third location on the core structure, wherein the anchor molecule is linked to the second anchor linker. In some embodiments, the anchor molecule comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor bridge comprises a polymer core. In some embodiments, the polymer core of the anchor bridge comprises a nucleic acid (DNA or RNA) of specific sequence or a polymer like PEG. In some embodiments, the third core linker, first anchor linker, second anchor linker, and anchor molecule independently comprise an anchor reactive molecule or DNA sequence domain. In some embodiments, each anchor reactive molecule independently comprises an amine, a thiol, a DBCO, a maleimide, biotin, an azide, an acrydite, a NHS-ester, a single stranded nucleic acid (RNA or DNA) of specific sequence, one or more polymers like PEG or polymerization initiators, or combinations thereof. In some embodiments, the anchor molecule is linked to the second anchor linker through a chemical bond. In some embodiments, the anchor molecule is covalently bonded to the second anchor linker. In some embodiments, the trigger further cleaves 1) the second anchor linker from the anchor molecule, 2) the first anchor linker from the third core linker, or combinations thereof. In some embodiments, the first and second locations are situated on a first side of the core structure, and the third location is situated on a second side of the core structure.

In some embodiments, the signal comprises the detector barcode, the capture barcode, or combinations thereof, corresponding to a supramolecular structure that shifted to a stable state. In some embodiments, the detector barcode from a corresponding detector molecule for a supramolecular structure that shifted to a stable state is configured to be separated from said corresponding detector molecule, such that the corresponding signal comprises the respective detector barcode for detection of the analyte molecule bound to said corresponding detector molecule. In some embodiments, the separated detector barcode provides a DNA signal corresponding to the analyte molecule bound to the respective detector molecule. In some embodiments, the separated detector barcode is configured to be analyzed using genotyping, qPCR, sequencing, or combinations thereof. In some embodiments, the capture and detector molecules for the supramolecular structure is configured for binding to one or more specific types of analyte molecules.

In some embodiments, the supramolecular structure comprises a prescribed shape, size, molecular weight, or combinations thereof, so as to reduce or eliminate cross-reactions with another supramolecular structure. In some embodiments, the supramolecular structure comprises a plurality of capture and detector molecules. In some embodiments, the supramolecular structure comprises a prescribed stoichiometry of the capture and detector molecules so as to reduce or eliminate cross-reactions with another supramolecular structure.

In some embodiments, the sample comprises a biological particle or a biomolecule. In some embodiments, the sample comprises an aqueous solution comprising a protein, a peptide, a fragment of a peptide, a lipid, DNA, RNA, an organic molecule, a viral particle, an exosome, an organelle, or any complexes thereof. In some embodiments, the sample comprises a tissue biopsy, blood, blood plasma, Urine, Saliva, Tear, Cerebrospinal fluid, extracellular fluid, cultures cells, culture media, discarded tissue, plant matter, a synthetic protein, a bacterial and/or viral sample or fungal tissue, or combinations thereof.

### BRIEF DESCRIPTION OF THE DRAWINGS

Specific embodiments of the disclosed devices, delivery systems, or methods will now be described with reference to the drawings. Nothing in this detailed description is intended to imply that any particular component, feature, or step is essential to the invention.

FIG. 1 depicts an exemplary depiction of a supramolecular structure and the related subcomponents.

FIG. 2 depicts an exemplary depiction of an assembled three-arm nucleic acid junction based supramolecular structure and related subcomponents.

FIG. 3 depicts an exemplary depiction of the individual subcomponents of the three-arm nucleic acid junction based supramolecular structure from FIG. 2.

FIG. 4 depicts an exemplary depiction of the deconstructor molecules corresponding to the subcomponents of the three-arm nucleic acid junction based supramolecular structure from FIG. 2.

FIG. 5 depicts an exemplary depiction of an assembled DNA origami based supramolecular structure and related subcomponents.

FIG. 6 depicts an exemplary depiction of the individual subcomponents of the DNA origami based supramolecular structure from FIG. 5.

FIG. 7 depicts an exemplary depiction of the deconstructor molecules corresponding to the subcomponents of the DNA origami based supramolecular structure from FIG. 5.

FIG. 8 provides an exemplary depiction of a supramolecular structure in an unstable state before and after being subject to a trigger (e.g., interaction with a deconstructor molecule).

FIG. 9 provides an exemplary depiction of a supramolecular structure in a stable state before and after being subject to a trigger (e.g., interaction with a deconstructor molecule).

FIG. 10 provides an exemplary depiction of a supramolecular structure shifting from an unstable state to a stable state after interaction with an analyte molecule, and the respective configurations before and after being subject to a trigger (e.g., interaction with a deconstructor molecule).

FIG. 11 provides an exemplary depiction of a supramolecular structure shifting from a stable state to an unstable state after interaction with an analyte molecule, and the respective configurations before and after being subject to a trigger (e.g., interaction with a deconstructor molecule).

FIG. 12 provides an exemplary depiction of a method for detecting and quantifying analyte molecules using a plurality of supramolecular structures.

FIG. 13 provides an exemplary depiction of a method for forming a hydrogel bead attached with a plurality of supramolecular structures.

FIG. 14 provides an exemplary depiction of a method for forming a hydrogel bead attached with a plurality of supramolecular structures, using droplet technology.

FIG. 15 provides an exemplary depiction of attaching a plurality of supramolecular structures onto a solid substrate (e.g., microparticle).

FIG. 16 provides an exemplary depiction of a method for detecting and quantifying analyte molecules using a plurality of supramolecular structures embedded within hydrogel beads.

FIG. 17 provides an exemplary depiction of trapping a single cell and supramolecular structures embedded within a hydrogel bead in a droplet, as part of a method for detecting and quantifying intracellular analyte molecules.

FIG. 18 provides an exemplary depiction of collecting and processing droplets enclosing a single cell and supramolecular structures embedded within a hydrogel bead, as part of a method for detecting and quantifying intracellular analyte molecules.

FIG. 19 provides an exemplary depiction of trapping supramolecular structures with captured intracellular analyte molecules (from FIG. 18) and barcoded beads in a droplet, as part of a method for detecting and quantifying intracellular analyte molecules.

FIG. 20 provides an exemplary depiction of collecting and processing droplets enclosing supramolecular structures with captured intracellular analyte molecules (from FIG. 18) and barcoded beads in a droplet, as part of a method for detecting and quantifying intracellular analyte molecules.

FIG. 21 provides an exemplary depiction of a method for detecting and quantifying analyte molecules using a plurality of supramolecular structures attached to a substrate.

FIG. 22 provides a depiction of an example microfluidic device from a side perspective, in accordance with aspects of the present techniques.

FIG. 23 provides a depiction of an example microfluidic device from a front perspective, in accordance with aspects of the present techniques.

FIG. 24 provides a depiction of an example of a substrate having a sample-facing surface on which one or more types of adapters are provided, in accordance with aspects of the present techniques.

FIG. 25 provides a depiction of a conjugated structure comprising a barcode element and flanking adaptor (e.g., primer) sequences, in accordance with aspects of the present techniques.

FIG. 26 provides a depiction of one example of a process flow comprising steps that may be performed to generate analyte capture data using spatial locations of capture sites, in accordance with aspects of the present techniques.

FIG. 27 provides a depiction of a further example of a process flow comprising steps that may be performed to generate analyte capture data, in accordance with aspects of the present techniques.

FIG. 28 provides a depiction of one example of a process flow comprising steps that may be performed to generate analyte capture data where analyte capture occurs during a solution phase, in accordance with aspects of the present techniques.

### DETAILED DESCRIPTION

Disclosed herein are structures and methods for detecting one or more analyte molecules present in a sample. In some embodiments, the one or more analyte molecules are detected using one or more supramolecular structures. In some embodiments, the one or more supramolecular structures are specifically designed to minimize cross-reactivity with each other. In some embodiments, the supramolecular structures are bi-stable, wherein the supramolecular structures shift from an unstable state to a stable state through interaction with one or more analyte molecules from the sample. In some embodiments, the stable state supramolecular structures are configured to provide a signal for analyte molecule detection and quantification. In some embodiments, the signal correlates to a DNA signal, such that detection and quantification of an analyte molecule comprises converting the presence of the analyte molecule into a DNA signal.

### Sample

In some embodiments, the sample comprises an aqueous solution comprising protein, peptides, peptide fragments, lipids, DNA, RNA, organic molecules, inorganic molecules, complexes thereof, or any combinations thereof. In some embodiments, the analyte molecules in the sample comprise protein, peptides, peptide fragments, lipids, DNA, RNA, organic molecules, inorganic molecules, complexes thereof, or any combinations thereof. In some embodiments, the analyte molecules comprise comprises intact proteins, denatured proteins, partially or fully degraded proteins, peptide fragments, denatured nucleic acids, degraded nucleic acid fragments, complexes thereof, or combinations thereof. In some embodiments, the sample is obtained from tissue, cells, the environment of tissues and/or cells, or combinations thereof. In some embodiments, the sample comprises tissue biopsy, blood, blood plasma, urine, saliva, a tear, cerebrospinal fluid, extracellular fluid, cultures cells, culture media, discarded tissue, plant matter, synthetic proteins, bacterial, viral samples, fungal tissue, or combinations thereof. In some embodiments, the sample is isolated from a primary source such as cells, tissue, bodily fluids (e.g., blood), environmental samples, or combinations thereof, with or without purification. In some embodiments, the cells are lysed using a mechanical process or other cell lysis methods (e.g., lysis buffer). In some embodiments, the sample is filtered using a mechanical process (e.g., centrifugation), micron filtration, chromatography columns, other filtration methods, or combinations thereof. In some embodiments, the sample is treated with one or more enzymes to remove one or more nucleic acids or one or more proteins. In some embodiments, the sample comprises intact proteins, denatured proteins, partially or fully degraded proteins, peptide fragments, denatured nucleic acids or degraded nucleic acid fragments. In some embodiments, the sample is collected from one or more individual persons, one or more animals, one or more plants, or combinations thereof. In some embodiments, the sample is collected from an individual person, animal and/or plant having a disease or disorder that comprises an infectious disease, an immune disorder, a cancer, a genetic disease, a degenerative disease, a lifestyle disease, an injury, a rare disease, an age-related disease, or combinations thereof.

### Supramolecular Structure

In some embodiments, the supramolecular structure is a programmable structure that can spatially organize molecules. In some embodiments, the supramolecular structure comprises a plurality of molecules linked together. In some embodiments, the plurality of molecules of the supramolecular structure interact with at least some of each other. In some embodiments, the supramolecular structure comprises a specific shape. In some embodiments, the supramolecular nanostructure comprises a prescribed molecular weight based on the plurality of molecules of the supramolecular structure. In some embodiments, the supramolecular structure is a nanostructure. In some embodiments the plurality of molecules are linked together through a bond, a chemical bond, a physical attachment, or combinations thereof. In some embodiments, the supramolecular structure comprises a large molecular entity, of specific shape and molecular weight, formed from a well-defined number of smaller molecules interacting specifically with each other. In some embodiments, the structural, chemical, and physical properties of the supramolecular structure are explicitly designed. In some embodiments, the supramolecular structure comprises a plurality of subcomponents that are spaced apart according to a prescribed distance. In some embodiments, at least a portion of the supramolecular structure is rigid. In some embodiments, at least a portion of the supramolecular structure is semi-rigid. In some embodiments, at least a portion of the supramolecular structure is flexible.

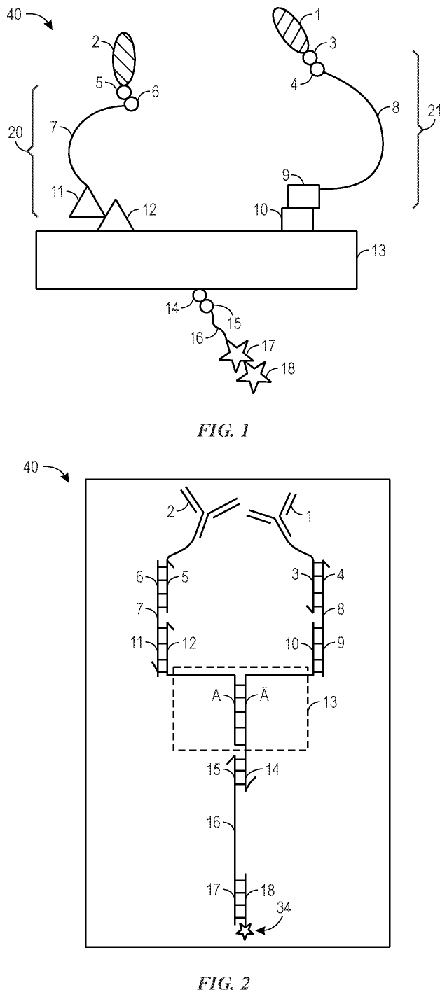

FIG. 1 provides an exemplary embodiment of a supramolecular structure 40 comprising a core structure 13, a capture molecule 2, a detector molecule 1, and an anchor molecule 18. In some embodiments, the supramolecular structure comprises one or more capture molecules 2, and one or more detector molecules 1 and optionally one or more anchor molecules 18. In some embodiments, the supramolecular structure does not comprise an anchor molecule. In some embodiments, the supramolecular structure is a polynucleotide structure.

In some embodiments, the core structure 13 comprises one or more core molecules linked together. In some embodiments, the one or more core molecules comprise 2, 3, 4, 5, 6, 7, 8, 9, 10, 20, 50, 100, 200 or 500 unique molecules that are linked together. In some embodiments, the one or more core molecules comprises from about 2 unique molecules to about 1000 unique molecules. In some embodiments, the one or more core molecules interact with each other and define the specific shape of the supramolecular structure. In some embodiments, the plurality of core molecules interact with each other through reversible non-covalent interactions. In some embodiments, the specific shape of the core structure is a three-dimensional (3D) configuration. In some embodiments, the one or more core molecules provide a specific molecular weight. In some embodiments, the core structure 13 is a nanostructure. In some cases, the one or more core molecules comprise one or more nucleic acid strands (e.g., DNA, RNA, unnatural nucleic acids), one or more branched nucleic acids, one or more peptides, one or more small molecules, or combinations thereof. In some embodiments, the core structure comprises a polynucleotide structure. In some embodiments, at least a portion of the core structure is rigid. In some embodiments, at least a portion of the core structure is semi-rigid. In some embodiments, at least a portion of the core structure is flexible. In some embodiments, the core structure comprises a scaffolded deoxyribonucleic acid (DNA) origami, a scaffolded ribonucleic acid (RNA) origami, a scaffolded hybrid DNA/RNA origami, a single-stranded DNA tile structure, a multi-stranded DNA tile structure, a single-stranded DNA origami, a single-stranded RNA origami, a single-stranded RNA tile structure, a multi-stranded RNA tile structures, a hierarchically composed DNA and/or RNA origami with multiple scaffolds, a peptide structure, or combinations thereof. In some embodiments, the DNA origami is scaffolded. In some embodiments, the RNA origami is scaffolded. In some embodiments, the hybrid DNA/RNA origami is scaffolded. In some embodiments, the core structure comprising a DNA origami, RNA origami, or hybrid DNA/RNA origami that comprises a prescribed two-dimensional (2D) or 3D shape.

As shown in FIG. 1, in some embodiments, the core structure 13 is configured to be linked to a capture molecule 2 (e.g., an affinity binder such as an antibody, aptamer, or nanobody), a detector molecule 1 (e.g., an affinity binder such as an antibody, aptamer, or nanobody), an anchor molecule 18 (e.g., an oligomer (e.g., oligonucleotide), such as a primer) for which a complementary fragment is grafted onto the surface of a substrate to facilitate binding of the supramolecular structure 40 to the surface), or combinations thereof. In some embodiments, the capture molecule 2, detector molecule 1, and/or anchor molecule 18 are immobilized with respect to the core nanostructure 13 when linked thereto. In some embodiments, any number of the one or more core molecules comprises one or more core linkers 10,12,14 configured to form a linkage with a capture molecule 2, a detector molecule 1, and/or an anchor molecule 18. In some embodiments, any number of the one or more core molecules are configured to be linked with one or more core linkers 10,12,14 that are configured to form a linkage with a capture molecule 2, a detector molecule 1, and/or an anchor molecule 18. In some embodiments, one or more core linkers are linked to one or more core molecules through a chemical bond. In some embodiments, at least one of the one or more core linkers comprises a core reactive molecule. In some embodiments, each core reactive molecule independently comprises an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, at least one of the one or more core linkers comprises a DNA sequence domain.

In some embodiments, the core structure 13 is linked to 1) a capture molecule 2 at a prescribed first location on the core structure, 2) a detector molecule 1 at a prescribed second location on the core structure, and optionally 3) an anchor molecule 18 at a prescribed third location on the core structure. In some embodiments, a specified first core linker 12 is disposed at the first location on the core structure, and a specified second core linker 10 is disposed at the second location on the core structure. In some embodiments, one or more core molecules at the first location are modified to form a linkage with the first core linker 12. In some embodiments, the first core linker 12 is an extension of the core structure 13. In some embodiments, one or more core molecules at the second location is modified to form a linkage with the second core linker 10. In some embodiments, the second core linker 10 is an extension of the core structure 13. In some embodiments, the 3D shape of the core structure 13 and relative distances of the first and second locations are specified to maximize the intramolecular interactions between the capture molecule 2 and detector molecule 1. In some embodiments, the 3D shape of the core structure 13 and relative distances of the first and second locations are specified to obtain a desired distance between the capture molecule 2 and detector molecule 1, so as to maximize the intramolecular interactions between the capture molecule 2 and detector molecule 1.

As described herein, in some embodiments, the distance between the capture molecule 2 and detector molecule 1 is about 3 nm, 4 nm, 5 nm, 6 nm, 10 nm, 12 nm, 15 nm, 20 nm, 30 nm, or 40 nm. In some embodiments, the distance between the capture molecule 2 and detector molecule 1 is about 1 nm to about 60 nm. In some embodiments, the distance between the capture molecule 2 and detector molecule 1 is about 1 nm to about 2 nm, about 1 nm to about 5 nm, about 1 nm to about 10 nm, about 1 nm to about 20 nm, about 1 nm to about 40 nm, about 1 nm to about 60 nm, about 2 nm to about 5 nm, about 2 nm to about 10 nm, about 2 nm to about 20 nm, about 2 nm to about 40 nm, about 2 nm to about 60 nm, about 5 nm to about 10 nm, about 5 nm to about 20 nm, about 5 nm to about 40 nm, about 5 nm to about 60 nm, about 10 nm to about 20 nm, about 10 nm to about 40 nm, about 10 nm to about 60 nm, about 20 nm to about 40 nm, about 20 nm to about 60 nm, or about 40 nm to about 60 nm, including increments therein. In some embodiments, the distance between the capture molecule 2 and detector molecule 1 is about 1 nm, about 2 nm, about 5 nm, about 10 nm, about 20 nm, about 40 nm, or about 60 nm. In some embodiments, the distance between the capture molecule 2 and detector molecule 1 is at least about 1 nm, about 2 nm, about 5 nm, about 10 nm, about 20 nm, or about 40 nm. In some embodiments, the distance between the capture molecule 2 and detector molecule 1 is at most about 2 nm, about 5 nm, about 10 nm, about 20 nm, about 40 nm, or about 60 nm.

In some embodiments, a specified third core linker 14 is disposed at the third location on the core structure 13. In some embodiments, one or more core molecules at the third location is modified to form a linkage with the third core linker 14. In some embodiments, the third core linker 12 is an extension of the core structure 13. In some embodiments, the first and second locations are disposed on a first side of the core structure 13, and the optional third location is disposed on a second side of the core structure 13.

In some embodiments, the capture molecule 2 comprises a protein, a peptide, an antibody, an aptamers (RNA and DNA), a fluorophore, a nanobody, a darpin, a catalyst, a polymerization initiator, a polymer like PEG, an organic molecule, or combinations thereof. In some embodiments, the detector molecule 1 comprises a protein, a peptide, an antibody, an aptamers (RNA and DNA), a fluorophore, a nanobody, a darpin, a catalyst, a polymerization initiator, a polymer like PEG, an organic molecule, or combinations thereof. In some embodiments, the anchor molecule comprises a reactive molecule. In some embodiments, the anchor molecule 18 comprises a reactive molecule. In some embodiments, the anchor molecule 18 comprises a DNA strand comprising a reactive molecule. In some embodiments, the anchor molecule comprises an oligomer (e.g., oligonucleotide, such as a primer) for which a complementary fragment is grafted onto the surface of a substrate to facilitate binding of the supramolecular structure (e.g., core structure 13) to the surface. In some embodiments, the anchor molecule 18 comprises an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the anchor molecule 18 comprises a protein, a peptide, an antibody, an aptamers (RNA and DNA), a flourophore, a nanobody, a darpin, a catalyst, a polymerization initiator, a polymer like PEG, an organic molecule or combinations thereof. In some embodiments, a single pair of a capture molecule 2 and corresponding detector molecule 1 is linked to the core structure 13. In some embodiments, a plurality of pairs of capture molecules 2 and corresponding detector molecules 1 are linked to a core structure 13. In some embodiments, the plurality of pairs of capture molecules 2 and corresponding detector molecules 1 are spaced apart from each other to minimize cross-talk, i.e., minimizing capture and/or detector molecules from a first pair interacting with capture and/or detector molecules from a second pair.

In some embodiments, each component of the supramolecular structure may be independently modified or tuned. In some embodiments, modifying one or more of the components of the supramolecular structure may modify the 2D and 3D geometry of the supramolecular structure itself. In some embodiments, modifying one or more of the components of the supramolecular structure may modify the 2D and 3D geometry of the core structure. In some embodiments, such capability for independently modifying the components of the supramolecular nanostructure enables precise control over the organization of one or more supramolecular structures on solid surfaces (e.g., planar surfaces or microparticles) and 3D volumes (e.g., within a hydrogel matrix).

### Capture Barcode

As shown in FIG. 1, in some embodiments, the capture molecule 2 is linked to the core structure 13 through a capture barcode 20. In some embodiments, the capture barcode 20 forms a linkage with the capture molecule 2, and the capture barcode 20 forms a linkage with the core structure 13. In some embodiments, the capture barcode 20 comprises a first capture linker 11, a second capture linker 6, and a capture bridge 7. In some embodiments, the first capture linker 11 comprises a reactive molecule. In some embodiments, the first capture linker 11 comprises a reactive molecule comprising an amine, a thiol, a DBCO, a NHS ester, a maleimide, an azide, an acrydite, a single stranded nucleic acid (e.g., RNA or DNA) of specific sequence, or a polymer (e.g., polyethylene glycol (PEG) or one or more polymerization initiators). In some embodiments, the first capture linker 11 comprises a DNA sequence domain. In some embodiments, the second capture linker 6 comprises a reactive molecule. In some embodiments, the second capture linker 6 comprises a reactive molecule comprising an amine, a thiol, a DBCO, a NHS ester, biotin, a maleimide, an azide, an acrydite, a single stranded nucleic acid (e.g., RNA or DNA) of specific sequence, or a polymer (e.g., polyethylene glycol (PEG) or one or more polymerization initiators). In some embodiments, the second capture linker comprises a DNA sequence domain. In some embodiments, the capture bridge 7 comprises a polymer. In some embodiments, the capture bridge 7 comprises a polymer that comprises a nucleic acid (e.g., DNA or RNA) of a specific sequence. In some embodiments, the capture bridge 7 comprises a polymer such as PEG. In some embodiments, the first capture linker 11 is attached to the capture bridge 7 at a first terminal end thereof, and the second capture linker 6 is attached to the capture bridge 7 at a second terminal end thereof. In some embodiments, the first capture linker 11 is attached to the capture bridge 7 via a chemical bond. In some embodiments, the second capture linker 6 is attached to the capture bridge 7 via a chemical bond. In some embodiments, the first capture linker 11 is attached to the capture bridge 7 via a physical attachment. In some embodiments, the second capture linker 6 is attached to the capture bridge 7 via a physical attachment.

In some embodiments, the capture barcode 20 is linked to the core structure 13 through a linkage between the first capture linker 11 and the first core linker 12. In some embodiments, as described herein, the first core linker 12 is disposed at a first location on the core structure 13. In some embodiments, the first capture linker 11 and first core linker 12 are linked together through a chemical bond. In some embodiments, the first capture linker 11 and first core linker 12 are linked together through a covalent bond. In some embodiments, the linkage between the first capture linker 11 and first core linker 12 is reversible upon being subjected to a trigger. In some embodiments, the trigger comprises interaction with a deconstructor molecule (“capture deconstructor molecule”, e.g., reference character 30 in FIGS. 4,7) or exposure to a trigger signal. In some embodiments, the capture deconstructor molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, the capture barcode 20 is linked to the capture molecule 2 through a linkage between the second capture linker 6 and a third capture linker 5 that is bound to the capture molecule 2. In some embodiments, the third capture linker 5 comprises a reactive molecule. In some embodiments, the third capture linker 5 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the third capture linker 5 comprises a DNA sequence domain. In some embodiments, the capture molecule 2 is bound to the third capture linker 5 through a chemical bond. In some embodiments, the capture molecule 2 is bound to the third capture linker 5 through a covalent bond. In some embodiments, the second capture linker 6 and third capture linker 5 are linked together through a chemical bond. In some embodiments, the second linker 6 and third capture linker 5 are linked together through a covalent bond. In some embodiments, the linkage between the second capture linker 6 and third capture linker 5 is reversible upon being subjected to a trigger. In some embodiments, the trigger comprises interaction with a deconstructor molecule (“capture barcode release molecule”, e.g., reference character 31 in FIGS. 4,7) or exposure to a trigger signal. In some embodiments, the capture barcode release molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, being subject to a trigger breaks the linkage between the first capture linker 11 and first core linker 12 only, thereby breaking the capture molecule linkage with the core nanostructure 13 at the first location. In some embodiments, the capture barcode 20, when separated from the core structure 13 and the capture molecule 2, is configured to provide a signal for detecting an analyte molecule. In some embodiments, the signal as provided from the capture barcode 20 is a DNA signal.

With the preceding in mind, and by way of characterizing one implementation of the capture bridge 7 and its use in a real-world context, in practice the capture bridge 7 may be implemented as or understood to be a DNA library element to which one or more universal adapters (e.g., primers) are conjugated. In such a context the DNA library may be the barcode (e.g., capture barcode 20) for the specific affinity binder (e.g., capture molecule 2, such as an antibody, aptamer, or nanobody) to which it is conjugated.

### Detector Barcode

As shown in FIG. 1, in some embodiments, the detector molecule 1 is linked to the core structure 13 through a detector barcode 21. In some embodiments, the detector barcode 21 forms a linkage with the detector molecule 1, and the detector barcode 21 forms a linkage with the core structure 13. In some embodiments, the detector barcode comprises a first detector linker 9, a second detector linker 4, and a detector bridge 8. In some embodiments, the first detector linker 9 comprises a reactive molecule. In some embodiments, the first detector linker 9 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the first detector linker 9 comprises a DNA sequence domain. In some embodiments, the second detector linker 4 comprises a reactive molecule. In some embodiments, the second detector linker 4 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the second detector linker 4 comprises a DNA sequence domain. In some embodiments, the detector bridge 8 comprises a polymer. In some embodiments, the detector bridge 8 comprises a polymer that comprises a nucleic acid (DNA or RNA) of a specific sequence. In some embodiments, the detector bridge 8 comprises a polymer such as PEG. In some embodiments, the first detector linker 9 is attached to the detector bridge 8 at a first terminal end thereof, and the second detector linker 4 is attached to the detector bridge 8 at a second terminal end thereof. In some embodiments, the first detector linker 9 is attached to the detector bridge 8 via a chemical bond. In some embodiments, the second detector linker 4 is attached to the detector bridge 8 via a chemical bond. In some embodiments, the first detector linker 9 is attached to the detector bridge 8 via a physical attachment. In some embodiments, the second detector linker 4 is attached to the detector bridge 8 via a physical attachment.

In some embodiments, the detector barcode 21 is linked to the core structure 13 through a linkage between the first detector linker 9 and the second core linker 10. In some embodiments, as described herein, the second core linker 10 is disposed at a second location on the core structure 13. In some embodiments, the first detector linker 9 and second core linker 10 are linked together through a chemical bond. In some embodiments, the first detector linker 9 and second core linker 10 are linked together through a covalent bond. In some embodiments, the linkage between the first detector linker 9 and second core linker 10 is reversible upon being subjected to a trigger. In some embodiments, the trigger comprises interaction with a deconstructor molecule (“detector deconstructor molecule”, e.g., reference character 28 in FIGS. 4,7) or exposure to a trigger signal. In some embodiments, the detector deconstructor molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, the detector barcode 21 is linked to the detector molecule 1 through a linkage between the second detector linker 4 and a third detector linker 3 bound to the detector molecule 1. In some embodiments, the third detector linker 3 comprises a reactive molecule. In some embodiments, the third detector linker 3 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the third detector linker 3 comprises a DNA sequence domain. In some embodiments, the detector molecule 1 is bound to the third detector linker 3 through a chemical bond. In some embodiments, the detector molecule 1 is bound to the third detector linker 3 through a covalent bond. In some embodiments, the second detector linker 4 and third detector linker 3 are linked together through a chemical bond. In some embodiments, the second detector linker 4 and third detector linker 3 are linked together through a covalent bond. In some embodiments, the linkage between the second detector linker 4 and third detector linker 3 is reversible upon being subjected to a trigger. In some embodiments, the trigger comprises interaction with a deconstructor molecule (“detector barcode release molecule”, e.g., reference character 29 in FIGS. 4,7) or exposure to a trigger signal. In some embodiments, the detector barcode release molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, being subject to a trigger breaks the linkage between the first detector linker 9 and second core linker 10 only, thereby breaking the detector molecule linkage with the core structure 13 at the second location. In some embodiments, the detector barcode 21, when separated from the core structure 13 and the detector molecule 1, is configured to provide a signal for detecting an analyte molecule. In some embodiments, the signal as provided from the detector barcode 21 is a DNA signal.

With the preceding in mind, and by way of characterizing one implementation of the detector bridge 8 and its use in a real-world context, in practice the detector bridge 8 may be implemented as or understood to be a DNA library element to which one or more universal adapters (e.g., primers) are conjugated. In such a context the DNA library may be the barcode (e.g., detector barcode 21) for the specific affinity binder (e.g., detector molecule 1, such as an antibody or nanobody) to which it is conjugated.

### Anchor Barcode

As shown in FIG. 1, in some embodiments, the anchor molecule 18 is linked to the core structure 13 through an anchor barcode. In some embodiments, the anchor barcode forms a linkage with the anchor molecule 18, and the anchor barcode forms a linkage with the core structure 13. In some embodiments, the anchor barcode comprises a first anchor linker 15, a second anchor linker 17, and an anchor bridge 16. In some embodiments, the first anchor linker 15 comprises a reactive molecule. In some embodiments, the first anchor linker 15 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the first anchor linker 15 comprises a DNA sequence domain. In some embodiments, the second anchor linker 17 comprises a reactive molecule. In some embodiments, the second anchor linker 17 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the second anchor linker 17 comprises a DNA sequence domain. In some embodiments, the anchor bridge 16 comprises a polymer. In some embodiments, the anchor bridge 16 comprises a polymer that comprises a nucleic acid (DNA or RNA) of a specific sequence. In some embodiments, the anchor bridge 16 comprises a polymer such as PEG. In some embodiments, the first anchor linker 15 is attached to the anchor bridge 16 at a first terminal end thereof, and the second anchor linker 17 is attached to the anchor bridge 16 at a second terminal end thereof. In some embodiments, the first anchor linker 15 is attached to the anchor bridge 16 via a chemical bond. In some embodiments, the second anchor linker 17 is attached to the anchor bridge 16 via a physical attachment. In some embodiments, the first anchor linker 15 is attached to the anchor bridge 16 via a chemical bond. In some embodiments, the second anchor linker 17 is attached to the anchor bridge 16 via a physical attachment.

In some embodiments, the anchor barcode is linked to the core structure 13 through a linkage between the first anchor linker 15 and the third core linker 14. In some embodiments, as described herein, the third core linker 14 is disposed at a third location on the core structure 13. In some embodiments, the first anchor linker 15 and third core linker 14 are linked together through a chemical bond. In some embodiments, the first anchor linker 15 and third core linker 14 are linked together through a covalent bond. In some embodiments, the linkage between the first anchor linker 15 and third core linker 14 is reversible upon being subjected to a trigger. In some embodiments, the trigger comprises interaction with a deconstructor molecule (“anchor deconstructor molecule”, e.g., reference character 32 in FIGS. 4,7) or exposure to a trigger signal. In some embodiments, the anchor deconstructor molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, the anchor barcode is linked to the anchor molecule 18 through a linkage between the second anchor linker 17 and the anchor molecule 18. As disclosed herein, in some embodiments, the anchor molecule comprises a reactive molecule, a reactive molecule, a DNA sequence domain, a DNA sequence domain comprising a reactive molecule, or combinations thereof. In some embodiments, the anchor molecule 18 is bound to the second anchor linker 17 through a chemical bond. In some embodiments, the anchor molecule 18 is bound to the second anchor linker 17 through a covalent bond. In some embodiments, the linkage between the second anchor linker 17 and anchor molecule 18 is reversible upon being subjected to a trigger. In some embodiments, the trigger comprises interaction with a deconstructor molecule (“anchor barcode release molecule” e.g., reference character 33 in FIGS. 4,7) or exposure to a trigger signal. In some embodiments, the anchor barcode release molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, being subject to a trigger breaks the linkage between the first anchor linker 15 and third core linker 14 only, thereby breaking the anchor molecule linkage with the core structure 13 at the third location.

In some embodiments, the capture deconstructor molecule, capture barcode release molecule, detector deconstructor molecule, and detector barcode release molecule comprise the same type of molecule. In some embodiments, the capture deconstructor molecule, capture barcode release molecule, detector deconstructor molecule, and detector barcode release molecule comprise different types of molecules. In some embodiments, the capture deconstructor molecule, capture barcode release molecule, detector deconstructor molecule, detector barcode release molecule, anchor deconstructor molecule, and anchor barcode release molecule comprise the same type of molecules. In some embodiments, the capture deconstructor molecule, capture barcode release molecule, detector deconstructor molecule, detector barcode release molecule, anchor deconstructor molecule, and anchor barcode release molecule comprise different types of molecules. In some embodiments, any combination of the capture deconstructor molecule, capture barcode release molecule, detector deconstructor molecule, detector barcode release molecule, anchor deconstructor molecule, and anchor barcode release molecule comprise the same type of molecules.

### Alternative Barcode Arrangements

While the preceding discussion describes the use of barcodes (e.g., barcode sequences) which may be associated with the capture molecule 2, detector molecule 1, and anchor molecule 18 respectively, it should be appreciated that other barcode arrangements and/or approaches are also contemplated. By way of example, instead of a separate, unique barcode associated with each of the capture molecule 2, detector molecule 1, and anchor molecule 18, in practice some or all of these molecules may be grouped together in relation to a respective unique barcode such that a given barcode may be associated with an overall supramolecular structure 40 having a respective, known combination of detector, capture, and anchor molecules. The barcode in this context may be associated with the supramolecular structure 40 itself rather than the linkage of the respective detector, capture, or anchor molecules. In accordance with this approach, presence of a barcode on a respective supramolecular structure 40 may be indicative of a given combination of detector and capture molecules, a given combination of detector, capture, and anchor molecules, a given combination of detector and anchor molecules, a given combination of capture and anchor molecules, and so forth, that are linked to the respective supramolecular structure 40. In this manner, detection of the given barcode associated with a supramolecular structure 40 may be used to infer the presence or function of a supramolecular structure 40 having the known combination of capture, detector, and/or anchor molecules.

In such a context, the linkage of the capture molecule 2, detector molecule 1, and/or anchor molecule 18 to the core structure 13 may be separated from the use of separate barcodes for each of these molecules. That is, the barcode sequence indicative of the respective capture molecule 2, detector molecule 1, and/or anchor molecule 18 (or combinations of these molecules) that are present on a respective supramolecular structure 40 may itself be present at another location or locations on the core structure 13. The barcode in this context is, therefore, not part of the linkage holding the respective capture molecule 2, detector molecule 1, or anchor molecule 18 to the core structure 13, but may instead be a distinct structure localized on the core structure 13. The molecular bridges connecting the capture molecule 2, detector molecule 1, and/or anchor molecule 18 to the core structure 13 in this example, therefore, may be linkage structures, still potentially unique to the respective molecules they attach, but without having identification or barcode functionality. It should be appreciated, however, that even if a separate barcode is employed that does not link the core structure 13 to the capture molecule 2, detector molecule 1, and/or anchor molecule 18, the detector barcode, capture barcode, and/or anchor barcode as discussed herein may still additionally be present as part of the respective linkage structure so as to facilitate various operational functions or operations. That is, use of a barcode separate from the linkage structures for purposes as discussed herein does not preclude the presence or use of other barcodes that are present as part of the linkage structures holding the capture molecule 2, detector molecule 1, and/or anchor molecule 18 to the core structure 13.

As may be appreciated, in a context as presently described where the barcode functionality is not limited to the linkage structures, more barcodes may be provided on the supramolecular structure 40 than there are linkage structure for capture molecules 2, detector molecules 1, and/or anchor molecules 18. That is, associating the barcode functionality to the respective linkage structures correspondingly limits the number of useful barcodes to the number of respective linkage structures (and therefore to the number of detector molecules, capture molecules, and/or anchor molecules). By providing the barcode functionality separate from the linkage functionality, more barcode sequences can be associated with a given supramolecular structure 40 than there are linkage structures on the respective supramolecular structure 40. By way of example, a given core structure 13 (e.g., a DNA origami core structure) may have hundreds (e.g., 200, 250, 300, 500) of locations suitable for attachment of molecular structures, such as oligomers used as linkage structures, but also suitable for barcode sequence attachment. In a context where a single detector molecule 1, capture molecule 2, and anchor molecule 18 are attached to the core structure 13, only three such sites are used, leaving some or all of the remainder of sites available for association with one or more barcode sequences that convey information about the supramolecular structure 40 (e.g., what detector and capture molecule are present, what detector, capture, and anchor molecule are present, and so forth). As discussed herein, this may provide benefits in terms of increased signal or signal-to-noise compared to contexts where the barcode sequences are limited to the linkage structures (though as noted above, such barcode sequences may still be provided as part of the linkage structures if so desired). It should also be appreciated that more than one type of barcode may be used to convey information about the supramolecular structure 40 in this manner. For example, while certain embodiments may employ barcodes that convey information in the aggregate about the supramolecular structure 40 (e.g., what capture and detector molecule are present), different barcodes may be associated with core structure 13 to provide information at whatever granularity is desired. For example, separate barcodes may be used to identify each of the detector and capture molecules, but such separate barcodes may still be attached at multiple sites on the core structure 13 so as to provide increased signal during processing relative to a single barcode associated with the linkage structures.

### Three Arm Nucleic Acid Junction Based Supramolecular Structure

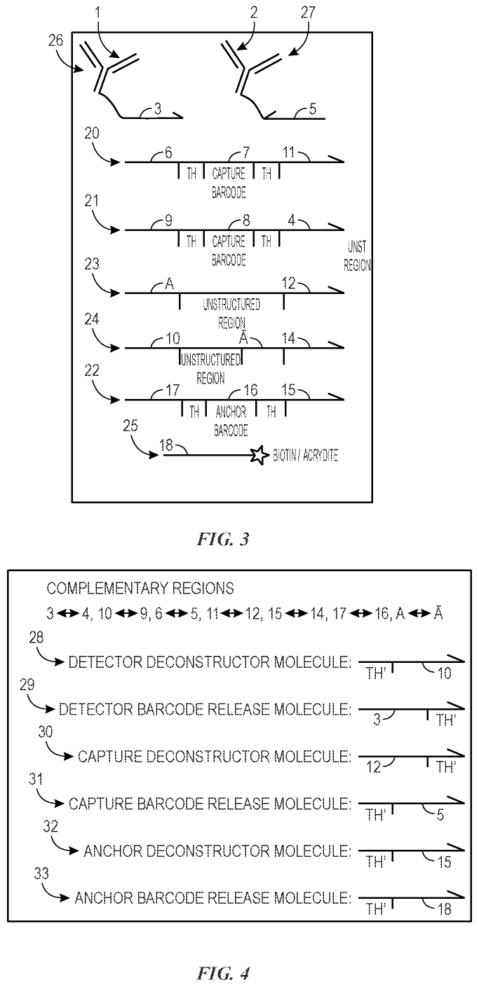

FIGS. 2-3 provides an exemplary depiction of a supramolecular structure 40 comprising a three arm nucleic acid junction and related subcomponents. FIG. 2 provides the complete supramolecular structure, while FIG. 3 provides the subcomponents that make up the supramolecular structure from FIG. 2. In some embodiments, the subcomponents of the supramolecular structure comprises five (5) DNA strands (ref. characters 20-24), one (1) DNA strand with a terminal modification 25, and two (2) antibodies (1,2) modified with a single DNA linker 3,5. FIG. 4 provides an exemplary depiction of the respective deconstructor molecules configured to cleave a respective subcomponent from the supramolecular structure 40 in FIG. 2. The references characters 1-18 in FIGS. 2-4 correspond to the respective components as provided with the same reference characters in FIG. 1.

As shown in FIGS. 2-3, in some embodiments of a supramolecular structure, the core structure comprises two strands, a first core strand 23 and a second core strand 24 that each comprise partially complementary DNA sequence domains labelled A and A respectively in the FIGS. 2-4.

In some embodiments, the first core strand 23 of the core structure comprises a first core linker 12 comprising a DNA sequence domain. In some embodiments, the first core strand 23 comprises the DNA sequence domain labelled as “A” in FIGS. 2-4, which is separated from the first core linker 12 by an unstructured DNA region. In some embodiments, the unstructured DNA region comprises a polymer spacer. In some embodiments, the polymer spacer comprises a nucleic acid (DNA or RNA) of a specific sequence. In some embodiments, the polymer spacer comprises a polymer such as PEG.

In some embodiments, the first core linker 12 is complementary to a first capture linker 11 on the capture barcode strand 20. In some embodiments, the capture barcode strand 20 comprises a DNA strand comprising the first capture linker 11 and a second capture linker 6 at either end of said capture barcode strand 20. In some embodiments, the first capture linker 11 comprises a DNA sequence domain. In some embodiments, the second capture linker 6 comprises a DNA sequence domain. In some embodiments, the capture barcode strand 20 further comprises a unique capture barcode sequence 7 in between the first and second capture linkers 11, 6. In some embodiments, the unique capture barcode sequence 7 comprises a nucleic acid (DNA or RNA) of a specific sequence. In some embodiments, the unique capture barcode sequence 7 comprises a polymer such as PEG. In some embodiments, the capture barcode 20 comprises a short domain called the toeholds (“TH”). In some embodiments, the capture barcode sequence 7 comprises the toeholds (“TH”).

In some embodiments, the second capture linker 6 is complementary to a third capture linker 5. In some embodiments, the third capture linker 5 is a DNA sequence domain. In some embodiments, a capture molecule 2 is bound 27 to the third capture linker 5. In some embodiments, the capture molecule 2 is covalently bound to the third capture linker 5. In some embodiments, the capture molecule 2 is a capture antibody.

In some embodiments, the second core strand 24 of the core structure comprises a second core linker 10 comprising a DNA sequence domain. In some embodiments, the second core strand 24 comprises the DNA sequence domain labelled as “A” in FIGS. 2-4, which is separated from the second core linker 10 by an unstructured DNA region. In some embodiments, the unstructured DNA region comprises a polymer spacer. In some embodiments, the polymer spacer comprises a nucleic acid (DNA or RNA) of a specific sequence. In some embodiments, the polymer spacer comprises a polymer such as PEG. In some embodiments, the second core strand 24 further comprises a third core linker 14 adjacent to the sequence domain “A”. In some embodiments, the third core linker 14 comprises a DNA sequence domain.

In some embodiments, the second core linker 10 is complementary to a first detector linker 9 on the detector barcode strand 21. In some embodiments, the detector barcode strand 21 comprises a DNA strand comprising the first detector linker 9 and a second detector linker 4 at either end of the detector barcode section 21. In some embodiments, the first detector linker 9 comprises a DNA sequence domain. In some embodiments, the second detector linker 4 comprises a DNA sequence domain. In some embodiments, the detector barcode strand 21 further comprises a unique detector barcode sequence 8 in between the first and second detector linkers 9, 4. In some embodiments, the unique detector barcode sequence 8 comprises a nucleic acid (DNA or RNA) of a specific sequence. In some embodiments, the unique detector barcode sequence 8 comprises a polymer such as PEG. In some embodiments, the detector barcode 21 comprises a short domain called the toeholds (“TH”). In some embodiments, the detector barcode sequence 8 comprises the toeholds (“TH”).

In some embodiments, the second detector linker 4 is complementary to a third detector linker 3. In some embodiments, the third detector linker 3 is a DNA sequence domain. In some embodiments, a detector molecule 1 is bound 26 to the third detector linker 3. In some embodiments, the detector molecule 1 is covalently bound to the third capture linker 3. In some embodiments, the detector molecule 1 is a detector antibody.

In some embodiments, the third core linker 14 is complementary to a first anchor linker 15 on the anchor barcode strand 22. In some embodiments, the anchor barcode strand 22 comprises a DNA strand comprising the first anchor linker 15 and a second anchor linker 17 at either end of the anchor barcode section 22. In some embodiments, the first anchor linker 15 comprises a DNA sequence domain. In some embodiments, the second anchor linker 17 comprises a DNA sequence domain. In some embodiments, the anchor barcode strand 22 further comprises a unique anchor barcode sequence 16 in between the first and second anchor linkers 15, 17. In some embodiments, the unique anchor barcode sequence 16 comprises a nucleic acid (DNA or RNA) of a specific sequence. In some embodiments, the unique anchor barcode sequence 16 comprises a polymer such as PEG. In some embodiments, the anchor barcode 22 comprises a short domain called the toeholds (“TH”). In some embodiments, the anchor barcode sequence 16 comprises the toeholds (“TH”).

In some embodiments, the second anchor linker 17 is complementary to the anchor molecule 18. In some embodiments, the anchor molecule 18 comprises a DNA sequence domain. In some embodiments, the anchor molecule 18 is linked 25 to a terminal modification 34. In some embodiments, the terminal modification 34 comprises a reactive molecule. In some embodiments, the terminal modification 34 comprises a reactive molecule. In some embodiments, the terminal modification 34 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators).

FIG. 4 provides an exemplary embodiment of deconstructor molecules that may be used to trigger different reactions on the supramolecular structure 40. In some embodiments, a detector deconstructor molecule 28 comprises of a TH′ domain, whose sequence is complementary to the TH domain on the detector barcode 21 and the second core linker 10 (e.g., a DNA sequence domain) on the second core strand 24. In some embodiments, the detector deconstructor molecule 28 is configured to cleave the link between the detector barcode 21 and the core structure (e.g., the second core strand 24). In some embodiments, a detector barcode release molecule 29 comprises of a TH′ domain, whose sequence is complementary to the TH domain on the detector barcode 21 and the third detector linker 3 (e.g., a DNA sequence domain). In some embodiments, the detector barcode release molecule 28 is configured to cleave the link between the detector barcode 21 and the detector molecule 1.

In some embodiments, a capture deconstructor molecule 30 comprises a TH′ domain, whose sequence is complementary to the TH domain on the capture barcode 20 and the first core linker 12 (e.g., a DNA sequence domain) on the first core strand 23. In some embodiments, a capture deconstructor molecule 30 is configured to cleave the link between the capture barcode 20 and the core structure (e.g., the first core strand 23). In some embodiments, a capture barcode release molecule 31 comprises a TH′ domain, whose sequence is complementary to the TH domain on the capture barcode 20, and the third capture linker 5 (e.g., a DNA sequence domain). In some embodiments, the capture barcode release molecule 31 is configured to cleave the link between the capture barcode 20 and the capture molecule 2.

In some embodiments, an anchor deconstructor molecule 32 comprises a TH′ domain, whose sequence is complementary to the TH domain on the anchor barcode 22 and third core linker 14 (e.g., a DNA sequence domain) on the second core strand. In some embodiments, the anchor deconstructor molecule 32 is configured to cleave the link between the anchor barcode 22 and the core structure (e.g., the second core strand 24). In some embodiments, an anchor barcode release molecule 33 comprises a “TH′” domain, whose sequence is complementary to the “TH” domain on the anchor barcode 22 and the anchor molecule 18 (e.g., a DNA sequence domain). In some embodiments, the anchor barcode release molecule 33 is configured to cleave the link between the anchor barcode 22 and the anchor molecule 18.

In some embodiments, each of the different DNA domain sequences (reference character 1, 2, 3, 4, 5, 6, 7, 8, 9, 10, 11, 12, 13, 14, 15, A, A, TH, Capture Barcode 20, Detector Barcode 21 and Anchor barcode 22) independently comprise nucleic acid sequences from about 2 nucleotides to about 80 nucleotides.

### DNA Origami Based Supramolecular Structure

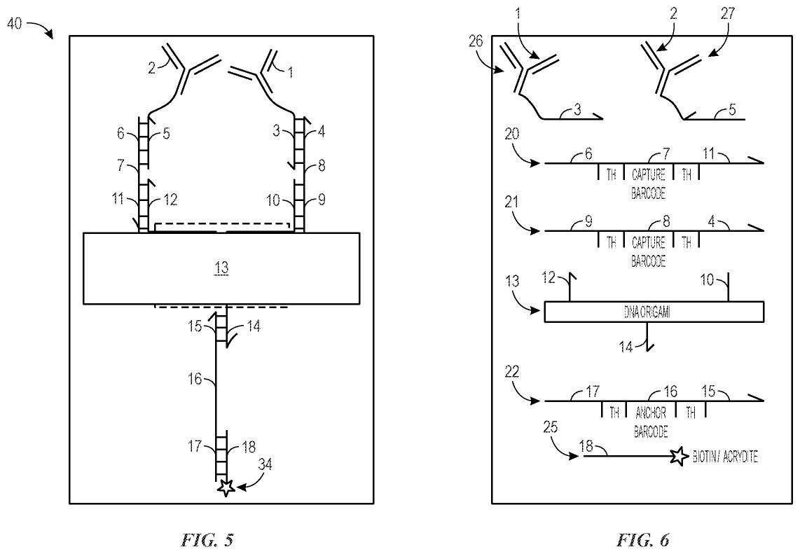

FIGS. 5-6 provides an exemplary depiction of a supramolecular structure 40 comprising a DNA origami and related subcomponents. FIG. 5 provides the complete supramolecular structure, while FIG. 6 provides the subcomponents that make up the supramolecular structure from FIG. 5. In some embodiments, the subcomponents of the supramolecular structure comprises a DNA origami 13 as a core structure, three (3) DNA strands (ref. characters 20-22), one (1) DNA strand with a terminal modification 25, and two (2) antibodies (1,2) modified with a single DNA linker 3,5. FIG. 6 provides an exemplary depiction of the respective deconstructor molecules configured to cleave a respective subcomponent from the supramolecular structure 40 in FIG. 5. The references characters 1-18 in FIGS. 5-7 correspond to the respective components as provided with the same reference characters in FIG. 1.

In some embodiments, the core structure 13 comprises a scaffolded DNA origami, wherein a circular ssDNA molecule, called “scaffold” strand, is folded into a predefined 2D or 3D shape by interacting with 2 or more short ssDNA, called “staple” strands, which interact with specific sub-sections of the ssDNA “scaffold” strand.

As shown in FIGS. 5-6, in some embodiments of a supramolecular structure, the core structure 13 comprises a DNA origami. In some embodiments, the core structure 13 comprises a first core linker 12 comprising a DNA sequence domain. In some embodiments, the first core linker 12 is complementary to a first capture linker 11 on the capture barcode strand 20. In some embodiments, the capture barcode strand 20 comprises a DNA strand comprising the first capture linker 11 and a second capture linker 6 at either end of said capture barcode strand 20. In some embodiments, the first capture linker 11 comprises a DNA sequence domain. In some embodiments, the second capture linker 6 comprises a DNA sequence domain. In some embodiments, the capture barcode strand 20 further comprises a unique capture barcode sequence 7 in between the first and second capture linkers 11, 6. In some embodiments, the unique capture barcode sequence 7 comprises a nucleic acid (DNA or RNA) of a specific sequence. In some embodiments, the unique capture barcode sequence 7 comprises a polymer such as PEG. In some embodiments, the capture barcode 20 comprises a short domain called the toeholds (“TH”). In some embodiments, the capture barcode sequence 7 comprises the toeholds (“TH”).

In some embodiments, the second capture linker 6 is complementary to a third capture linker 5. In some embodiments, the third capture linker 5 is a DNA sequence domain. In some embodiments, a capture molecule 2 is bound 27 to the third capture linker 5. In some embodiments, the capture molecule 2 is covalently bound to the third capture linker 5. In some embodiments, the capture molecule 2 is a capture antibody.

In some embodiments, the core structure 13 comprises a second core linker 10 comprising a DNA sequence domain. In some embodiments, the second core linker 10 is complementary to a first detector linker 9 on the detector barcode strand 21. In some embodiments, the detector barcode strand 21 comprises a DNA strand comprising the first detector linker 9 and a second detector linker 4 at either end of the detector barcode section 21. In some embodiments, the first detector linker 9 comprises a DNA sequence domain. In some embodiments, the second detector linker 4 comprises a DNA sequence domain. In some embodiments, the detector barcode strand 21 further comprises a unique detector barcode sequence 8 in between the first and second detector linkers 9, 4. In some embodiments, the unique detector barcode sequence 8 comprises a nucleic acid (DNA or RNA) of a specific sequence. In some embodiments, the unique detector barcode sequence 8 comprises a polymer such as PEG. In some embodiments, the detector barcode 21 comprises a short domain called the toeholds (“TH”). In some embodiments, the unique detector barcode sequence 8 comprises the toeholds (“TH”).

In some embodiments, the second detector linker 4 is complementary to a third detector linker 3. In some embodiments, the third detector linker 3 is a DNA sequence domain. In some embodiments, a detector molecule 1 is bound 26 to the third detector linker 3. In some embodiments, the detector molecule 1 is covalently bound to the third capture linker 3. In some embodiments, the detector molecule 1 is a detector antibody.

In some embodiments, the core structure 13 comprises a third core linker 14 that comprises a DNA sequence domain. In some embodiments, the third core linker 14 is complementary to a first anchor linker 15 on the anchor barcode strand 22. In some embodiments, the anchor barcode strand 22 comprises a DNA strand comprising the first anchor linker 15 and a second anchor linker 17 at either end of the anchor barcode section 22. In some embodiments, the first anchor linker 15 comprises a DNA sequence domain. In some embodiments, the second anchor linker 17 comprises a DNA sequence domain. In some embodiments, the anchor barcode strand 22 further comprises a unique anchor barcode sequence 16 in between the first and second anchor linkers 15, 17. In some embodiments, the unique detector barcode sequence 16 comprises a nucleic acid (DNA or RNA) of a specific sequence. In some embodiments, the unique detector barcode sequence 16 comprises a polymer such as PEG. In some embodiments, the anchor barcode 22 comprises a short domain called the toeholds (“TH”). In some embodiments, the anchor barcode sequence 16 comprises the toeholds (“TH”).

In some embodiments, the second anchor linker 17 is complementary to the anchor molecule 18. In some embodiments, the anchor molecule 18 comprises a DNA sequence domain. In some embodiments, the anchor molecule 18 is linked 25 to a terminal modification 34. In some embodiments, the terminal modification 34 comprises a reactive molecule. In some embodiments, the terminal modification 34 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators).

FIG. 6 provides an exemplary embodiment of deconstructor molecules that may be used to trigger different reactions on the supramolecular structure 40. In some embodiments, a detector deconstructor molecule 28 comprises of a TH′ domain, whose sequence is complementary to the TH domain on the detector barcode 21 and the second core linker 10 (e.g., a DNA sequence domain) on the core nanostructure 13. In some embodiments, the detector deconstructor molecule 28 is configured to cleave the link between the detector barcode 21 and the core structure 13. In some embodiments, a detector barcode release molecule 29 comprises of a TH′ domain, whose sequence is complementary to the TH domain on the detector barcode 21 and the third detector linker 3 (e.g., a DNA sequence domain). In some embodiments, the detector barcode release molecule 28 is configured to cleave the link between the detector barcode 21 and the detector molecule 1.

In some embodiments, a capture deconstructor molecule 30 comprises a TH′ domain, whose sequence is complementary to the TH domain on the capture barcode 20 and the first core linker 12 (e.g., a DNA sequence domain) on the core nanostructure 13. In some embodiments, a capture deconstructor molecule 30 is configured to cleave the link between the capture barcode 20 and the core structure 13. In some embodiments, a capture barcode release molecule 31 comprises a TH′ domain, whose sequence is complementary to the TH domain on the capture barcode 20, and the third capture linker 5 (e.g., a DNA sequence domain). In some embodiments, the capture barcode release molecule 31 is configured to cleave the link between the capture barcode 20 and the capture molecule 2.

In some embodiments, an anchor deconstructor molecule 32 comprises a TH′ domain, whose sequence is complementary to the TH domain on the anchor barcode 22 and third core linker 14 (e.g., a DNA sequence domain) on the core nanostructure 13. In some embodiments, the anchor deconstructor molecule 32 is configured to cleave the link between the anchor barcode 22 and the core structure 13. In some embodiments, an anchor barcode release molecule 33 comprises a TH′ domain, whose sequence is complementary to the TH domain on the anchor barcode 22 and the anchor molecule 18 (e.g., a DNA sequence domain). In some embodiments, the anchor barcode release molecule 33 is configured to cleave the link between the anchor barcode 22 and the anchor molecule 18.

### Stable and Unstable State of Supramolecular Structure

In some embodiments, the supramolecular structure comprises one or more stable state configurations. In some embodiments, the supramolecular structure comprises one or more unstable state configurations. In some embodiments, the supramolecular structure comprises a bi-stable configuration having a stable state configuration and an unstable state configuration. In some embodiments, the two states, stable and unstable are defined based on the ability of an individual supramolecular structure to remain structurally intact when subjected to a unique molecule (e.g., a deconstructor molecule) and/or a trigger signal. In some embodiments, when the supramolecular structure is in the stable state, then all the different components that are part of the supramolecular structure remain physically connected to each other even after being exposed to the deconstructor molecule and/or trigger signal. In some embodiments, when the supramolecular structure is in the unstable state, then the exposure to the deconstructor molecule and/or trigger signal leads to a defined section (e.g., one or more subcomponents) of the supramolecular structure being physically cleaved, i.e., unbound (separated) from the supramolecular structure. In some embodiments, the supramolecular structure is configured to shift from a stable state to an unstable state upon interaction with an analyte molecule (as described herein). In some embodiments, the supramolecular structure is configured to shift from a unstable state to a stable state upon interaction with an analyte molecule (as described herein). In some embodiment, the analyte molecule that triggers the state change of the supramolecular structure comprises a protein, clusters of proteins, peptide fragments, cluster of peptide fragments, DNA, RNA, DNA nanostructure, RNA nanostructures, lipids, an organic molecule, an inorganic molecule, or any combination thereof.

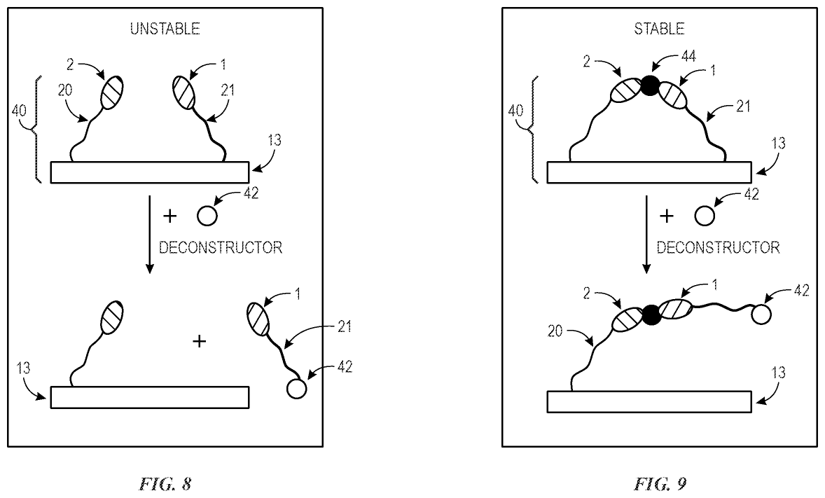

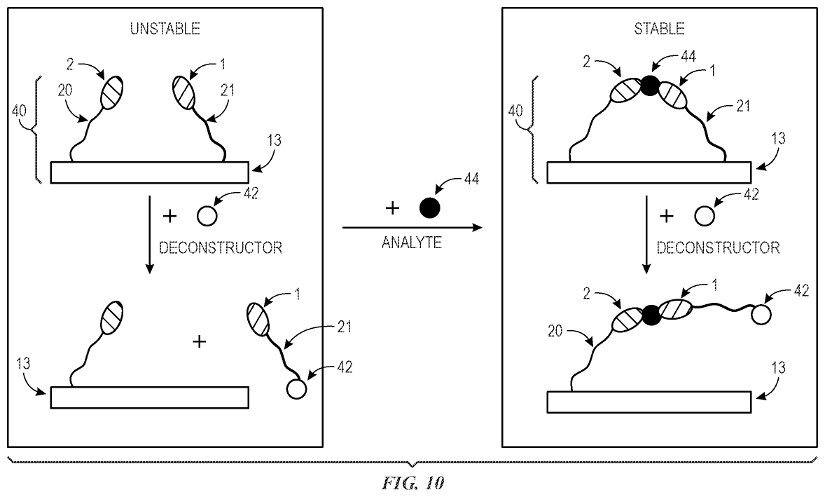

In some embodiments, a supramolecular structure in an unstable state configuration comprises a physical state wherein a linkage between the core structure 13 and a capture molecule 2 may be cleaved such that the capture molecule 2 is unbound from the core nanostructure 13. In some embodiments, the unstable state configuration comprises a physical state wherein a linkage between the core nanostructure 13 and a detector molecule 1 may be cleaved such that the detector molecule 1 is unbound from the core nanostructure 13. In some embodiments, the unstable state configuration comprises a physical state wherein a linkage between the core nanostructure 13 and a capture molecule 2 and a linkage between the core nanostructure 13 and a detector molecule 1 may be cleaved such that the capture molecule 2 and detector molecule 1 are unbound from the core nanostructure 13. In some embodiments, the linkage between the core nanostructure 13 and 1) the capture molecule 2, 2) the detector molecule 1, or 3) both, are cleaved upon being subjected to a trigger (e.g., a deconstructor molecule as described herein or trigger signal as described herein). FIG. 8 provides an exemplary depiction of a supramolecular structure 40 in an unstable state, wherein the detector molecule 1 is initially bound to the core structure 13 via a linkage with the detector barcode 21. With continued reference to FIG. 8, interaction with a deconstructor molecule 42 (e.g., detector deconstructor molecule 28) subsequently cleaves the linkage between the detector barcode 21 and core structure 13, such that the detector molecule 1 is unbound from the core nanostructure 13. In some embodiments, in the unstable state, the capture molecule 2 and detector molecule 1 on the core nanostructure 13 are freely diffusing with respect to each other, constrained only by the physical configuration of the core nanostructure 13.

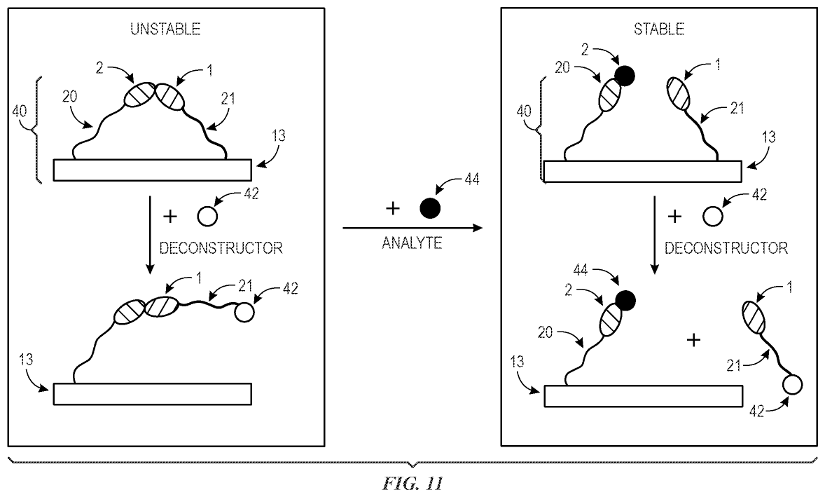

In some embodiments, the stable state configuration comprises a physical state wherein the capture molecule 2 remains bound to the core nanostructure 13 upon cleavage of a linkage between the core structure 13 and the capture molecule 2. In some embodiments, the stable state configuration comprises a physical state wherein the detector molecule 1 remains bound to the core structure 13 upon cleavage of a linkage between the core nanostructure 13 and the detector molecule 1. In some embodiments, the stable state configuration comprises a physical state wherein the capture molecule 2 and detector molecule 1 are proximally positioned with respect to each other. In some embodiments, the detector molecule 1 and capture molecule 2 are proximally positioned with respect to each other with, or without, explicit bond formation between each other. In some embodiments, the detector 1 and capture 2 molecules are linked to each other. In some embodiments, the detector 1 and capture 2 molecules are linked to each other through a chemical bond. In some embodiments, the detector 1 and capture 2 molecules are linked together through a linkage with another molecule located between the capture and detector molecules (e.g., a sandwich formation). In some embodiments, the detector and capture molecules are linked together through linkage with an analyte molecule 44 from a sample (as described herein). FIG. 9 provides an exemplary depiction of a supramolecular structure 40 in a stable state, wherein the capture molecule 2 is linked to the detector molecule 1 through linkage with an analyte molecule 44. With continued reference to FIG. 9, interaction with a deconstructor molecule 42 cleaves the linkage between the detector molecule 1 and core structure 13, but the detector molecule 1 remains bound to the core nanostructure 13 through the linkage with the capture molecule 2. As described further herein, in some embodiments, a capture and/or detector molecule is configured to form a linkage with one or more specific types of analyte molecule from the sample. In some embodiments, interaction with the deconstructor molecule and/or trigger signal does not cleave the linkage between the capture and detector molecules.

FIG. 10 provides an exemplary embodiment of a supramolecular structure shifting from an unstable state to a stable state. As described herein, a supramolecular structure 40 in an unstable state configuration will be separated from a detector molecule 1 (the detector molecule will be unbound from the supramolecular structure) upon interaction with a corresponding deconstructor molecule 42 (e.g., detector deconstructor molecule 28) and/or a trigger signal. With continued reference to FIG. 10, in some embodiments, interaction with an analyte molecule 44 from a sample binds the capture molecule and detector molecule together with the analyte molecule located therebetween (e.g., a sandwich formation), thereby shifting the supramolecular structure 40 from an unstable state to a stable state. In some embodiments, the analyte molecule 44 comprises a single molecule. In some embodiments, the analyte molecule instead comprises a plurality of analyte molecules. In some embodiments, the analyte molecule instead comprises a molecular cluster. In some embodiments, as described herein and shown in FIG. 10, with the supramolecular structure in a stable state, interaction with a corresponding deconstructor molecule cleaves the linkage between the core structure 13 and the detector barcode 21, wherein the detector molecule 1 remains linked to the core structure 13 through the linkage with the capture molecule 2 and analyte molecule 44.

FIG. 11 provides an exemplary embodiment of a supramolecular structure 40 shifting from a stable state to an unstable state. As described herein, the supramolecular structure 40 is in a stable state configuration wherein the detector molecule 1 will remain linked to the core structure 13 upon interaction with a corresponding deconstructor molecule and/or trigger signal, due to the detector molecule 1 being linked to the capture molecule 2. With continued reference to FIG. 11, in some embodiments, interaction with an analyte molecule 44 from the sample cleaves the linkage between the capture molecule 2 and detector molecule 1, such that the analyte molecule 44 is bound to the capture molecule 1 only, thereby moving the supramolecular structure to an unstable state wherein the detector molecule 1 is bound to the core nanostructure 13 only through the linkage with the detector barcode 21. In some embodiments, the analyte molecule 44 comprises a single molecule. In some embodiments, the analyte molecule instead comprises a plurality of analyte molecules. In some embodiments, the analyte molecule instead comprises a molecular cluster. In some embodiments, as described herein and shown in FIG. 11, with the supramolecular structure in an unstable state, interaction with a corresponding deconstructor molecule 42 cleaves the linkage between the core structure 13 and the detector barcode 21, such that the detector molecule 1 is unbound (separated) from the core structure 13.

In some embodiments, the supramolecular structure 40 moves from a stable state to an unstable state upon interaction with an analyte molecule 44 that cleaves the linkage between a capture molecule 2 and detector molecule 1, wherein the analyte molecule 44 binds with the detector molecule 1. The capture molecule 2 is thereby unbound from the core structure 13 upon interaction with a corresponding destructor molecule 42 (e.g., capture deconstructor molecule 30).

### Methods for Detecting Analyte Molecules

As described herein, in some embodiments, one or more supramolecular structures enable the detection of one or more analyte molecules in a sample. In some embodiments, the supramolecular structure convert information about the presence of a given analyte molecule in a sample to a DNA signal. In some embodiments, the DNA signal corresponds to a capture barcode or detector barcode located on a supramolecular structure, wherein the capture molecule and detector molecule are simultaneously linked to the analyte molecule (e.g., sandwich formation). In some embodiments, capture and/or detector barcodes located on any unstable supramolecular structures are unbound therefrom using a trigger, such as a deconstructor molecule and/or a trigger signal. In some embodiments, the DNA signal is sequenced accordingly, and subsequently identified and correlated with the specific analyte molecule.

In some embodiments, detecting the presence of an analyte molecule, as described herein, comprises controllably releasing a single, or multiple, unique nucleic acid molecules into the solution to be used to identify as well as quantify properties of the analyte molecule from the sample that triggered the state change of the supramolecular structure. In some embodiments, said unique nucleic acid molecules are provided by capture barcodes and/or detector barcodes of the respective supramolecular structures. In some embodiments, detecting the presence of an analyte molecule, as described herein, comprises creating an optical or electrical signal connected to the state change that can be counted to quantify the concentration of the analyte molecule in solution.

In some embodiments, a plurality of analyte molecules are simultaneously detected in a sample through multiplexing, wherein a plurality of supramolecular structures provide a plurality of signals (e.g., detector barcode, capture barcode) for sequencing and analyte identification. In some embodiments, methods described herein for detecting analytes in a sample provide a high-throughput and high-multiplexing capability by using a plurality of supramolecular structures. In some embodiments, the high-throughput and high-multiplexing capability provides high accuracy for analyte molecule detection and quantification. In some embodiments, methods described herein for detecting analytes in a sample are configured to characterize and/or identify biopolymers, including proteins molecules, quickly and at high sensitivity and reproducibility. In some embodiments, the plurality of supramolecular structures are configured to limit cross-reactivity associated errors. In some embodiments, such cross-reactivity associated errors comprise capture and/or detector molecules of a supramolecular structure interacting with capture and/or detector molecules of another supramolecular structure (e.g., intermolecular interactions). In some embodiments, each core structure of the plurality of supramolecular structures is identical to one another. In some embodiments, the structural, chemical, and physical property of each supramolecular structure is explicitly designed. In some embodiments, identical core structures have a prescribed shape, size, molecular weight, prescribed number of capture and detector molecules, predetermined distance between corresponding capture and detector molecules (as described herein), prescribed stoichiometry between corresponding capture and detector molecules, or combinations thereof, so as to limit the cross-reactivity between supramolecular structures. In some embodiments, the molecular weight of every core structure is identical and precise up to the purity of the core molecules. In some embodiments, each core structure has at least one capture molecule and at least one corresponding detector molecule.

In some embodiments, the plurality of supramolecular structures independently interact with different analyte molecules from a sample since the state change (from unstable to stable) is driven primarily by intramolecular interaction (capture and detector molecules on the same supramolecular structure). In some embodiments, the plurality of supramolecular structures might share structural similarities due to certain subcomponents being the same, however the interaction between an analyte molecule from the sample and supramolecular structure is defined by the corresponding capture molecule and detector molecule. In some embodiments, each pair of detector and capture molecules on a given supramolecular structure may specifically interact with a particular analyte molecule in the sample, leading to a state change of supramolecular structure upon interacting with the particular analyte molecule. In some embodiments, each supramolecular structure comprises unique DNA barcodes corresponding to the respective pair of detector and capture molecules. In some embodiments, a pair of detector and capture molecules on a given supramolecular structure is designed to interact with more than one analyte molecule in the sample.

In some embodiments, each supramolecular structure is configured for single-molecule sensitivity to ensure the highest possible dynamic range needed to quantitatively capture the wide range of molecular concentrations within a typical complex biological sample. In some embodiments, single-molecule sensitivity comprises the capture and detector molecules of a given supramolecular structure configured to shift from an unstable state to a stable state (or vice versa) through binding with a single analyte molecule. In some embodiments, the plurality of supramolecular structures limit or eliminate the manipulation of the sample needed to reduce non-specific interaction as well as any user induced errors.

In some embodiments, the plurality of supramolecular structures are provided in a solution. In some embodiments, the plurality of supramolecular structures are attached to one or more substrates. In some embodiments, the plurality of supramolecular structures are attached to one or more widgets. In some embodiments, the plurality of supramolecular structures are attached to one or more solid substrates, one or more polymer matrices, one or more molecular condensates, or combinations thereof. In some embodiments, the one or more polymer matrices comprises one or more hydrogel particles. In some embodiments, the one or more polymer matrices comprises one or more hydrogel beads. In some embodiments, the one or more solid substrates comprises one or more planar substrates. In some embodiments, the one or more solid substrates comprises one or more microbeads. In some embodiments, the one or more solid substrates comprises one or more microparticles.

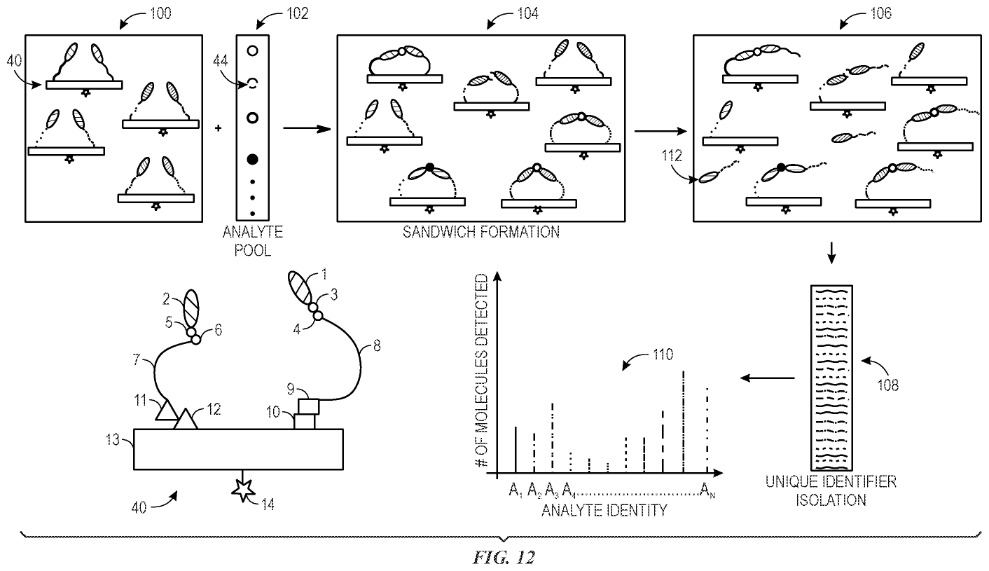

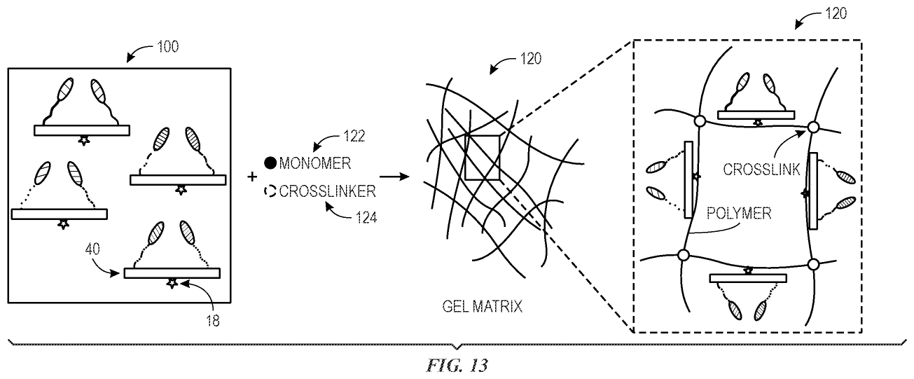

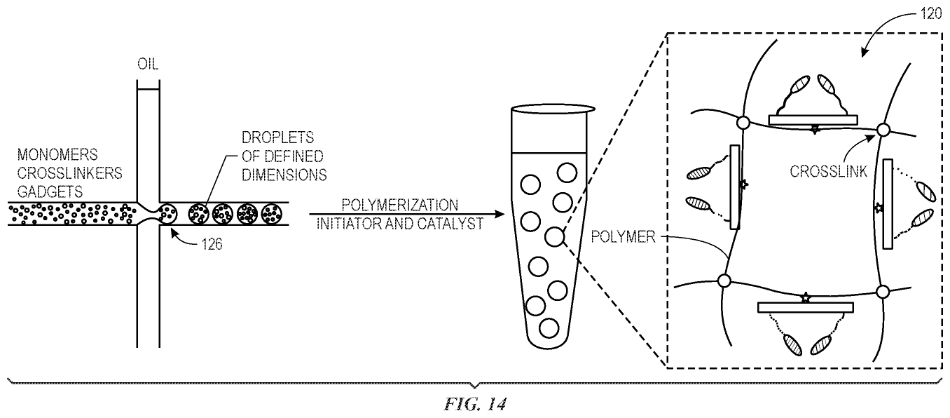

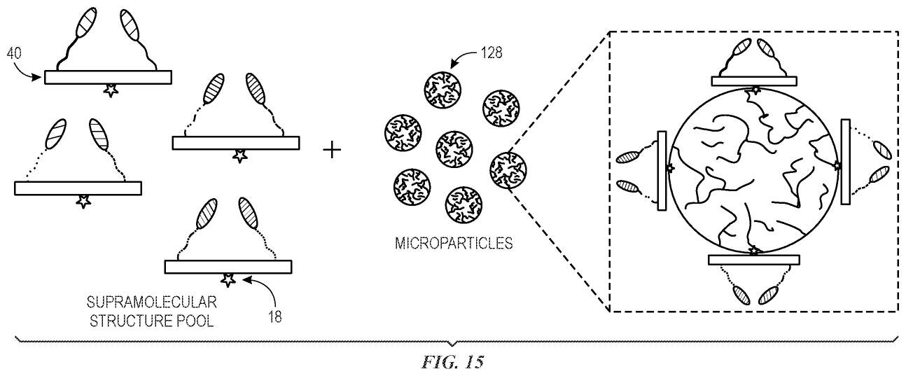

FIG. 12 provides an exemplary method for detecting one or more analyte molecules in a sample using one or more supramolecular structures. In some embodiments, the sample, comprising one or more analytes (e.g., analyte pool 102) is contacted with the one or more supramolecular structures 40 (e.g., supramolecular structure pool 100). In some embodiments, the supramolecular structures are attached to a plurality of widgets. In some embodiments, as described herein, the plurality of supramolecular structures are provided as being attached to one or more solid substrates, one or more polymer matrices, one or more molecular condensates, or combinations thereof. FIGS. 13-14 provide examples of supramolecular structures attached to a hydrogel bead (e.g., supramolecular structures embedded within a hydrogel bead). FIG. 15 provides an example of supramolecular structures attached to a solid substrate, e.g., a microparticle. In some embodiments, the sample comprises an aqueous solution, and is mixed with the supramolecular structures to form a combined solution. In some embodiments, contacting the sample with the supramolecular structures comprises incubating the sample with the supramolecular structures. In some embodiments, the sample and supramolecular structures are incubated in an incubator with prescribed environmental conditions. In some embodiments, the sample is incubated with the supramolecular structures for a time period from about 30 seconds to about 24 hours. In some embodiments, the sample is incubated with the supramolecular structures for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

With continued reference to FIG. 12, in some embodiments, the supramolecular structures are all in an unstable state (as shown with reference character 100). In some embodiments, and as described herein, interaction between an analyte molecule and corresponding capture 2 and detector 1 molecules shifts a respective supramolecular structure from an unstable state to a stable state (e.g., a sandwich formation with the capture molecule, analyte molecule, and detector molecule as shown with reference character 104). In some embodiments, a particular type of analyte molecule will bind with a particular pair of capture and detector molecules. In some embodiments, a given pair of capture and detector molecules are configured to bind with more than one type of analyte molecule. In some embodiments, the switching from an unstable state to a stable state for any given supramolecular structure is dependent on the specific capture and detector molecules bound thereto and the analyte molecules in the sample. In some embodiments, given that the state-change of the supramolecular structure is primarily dependent on the intra-molecular interactions (components located on the supramolecular structure), potential inter-molecular interactions between two different supramolecular structures are minimized or eliminated by limiting the net concentration of the supramolecular structures in the combined solution, such that the mean distance between any two supramolecular structures is larger than maximum intramolecular distance between a pair of capture and detector molecules on a given supramolecular structure.

As seen in FIG. 12, reference character 104, after contacting the sample, at least one of the supramolecular structures shifted to a stable state through interaction with an analyte molecule (e.g., sandwich formation wherein the capture molecule, analyte molecule and detector molecule are simultaneously linked together), while at least one of the supramolecular structures remained in an unstable state as the respective capture and detector molecules did not bind or interact with an analyte molecule from the sample.

After the sample has been contacted with the supramolecular structures for a prescribed amount of time, the combined solution of the sample and supramolecular structures, as shown in FIG. 12, is subjected to a trigger so as to cleave a linkage between the detector molecule and the core structure (reference character 106). In some embodiments, the trigger comprises introducing a solution comprising one or more deconstructor molecules (e.g., detector deconstructor molecule, reference character 28 from FIGS. 4,7) to the combined solution. In some embodiments, the trigger comprises subjecting the combined solution to a trigger signal. In some embodiments, the trigger comprises a combination of introducing a deconstructor molecule in the combined solution and subjecting the combined solution to a trigger signal. In some embodiments, as described herein, the deconstructor molecule comprises a nucleic acid (DNA or RNA), a peptide, a small organic molecule, or combinations thereof. In some embodiments, as described herein, the trigger signal comprises an electrical signal, microwave signal, ultraviolet illumination, visible illumination or near infra-red illumination. In some embodiments, the combined solution is subjected to the trigger for a prescribed amount of time. In some embodiments, the combined solution is incubated with one or more deconstructor molecules for a prescribed amount of time. In some embodiments, the combined solution is incubated with the deconstructor molecules for a time period from about 30 seconds to about 24 hours. In some embodiments, the combined solution is incubated with the deconstructor molecules for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

As shown in FIG. 12 reference character 106, in some embodiments, subjecting the combined solution to the trigger cleaves a linkage between the detector molecule and core structure of a supramolecular structure, such as the linkage between a detector barcode (e.g., reference character 21 from FIG. 1) and the core structure 13. In some embodiments, the cleavage is achieved through nucleic acid (DNA/RNA) strand displacement, optical cleavage, chemical cleavage, another technique known in the art, or combinations thereof. For supramolecular structures that shifted to a stable state, the detector molecule 1 is shown as remaining to be linked to the core structure 13 via linkage with the corresponding capture molecule 2. For supramolecular structures that remained in an unstable state, the detector molecule is shown as being unbound 112 from the respective supramolecular structure. In some embodiments, the unbound detector molecules 1 remain linked to the respective detector barcodes 21.

In some embodiments, the unbound detector molecules 1 (and corresponding detector barcode 21) are further separated from the combined solution. In some embodiments, the unbound detector molecules are separated from the combined solution through polyethylene glycol (PEG) precipitation. In some embodiments, the unbound detector molecules are separated from the combined solution by binding each core structure in the combined solution to microbeads, a solid support and/or magnetic beads through a corresponding anchor molecule on the respective core structure, followed by separation of the unbound detector molecules through centrifugation, micron filtration, chromatography or combinations thereof.

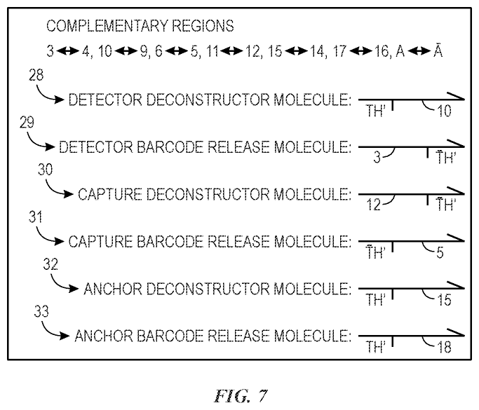

In some embodiments, after the unbound detector molecules have been separated from the combined solution, the detector barcodes 21 are cleaved from the corresponding detector molecules that are linked to a respective capture molecule (e.g., as located on a supramolecular structure that shifted to a stable state). In some embodiments, the detector barcodes 21 are cleaved from the corresponding detector molecules through nucleic acid (DNA/RNA) strand displacement, optical cleavage, chemical cleavage, or a combination thereof. In some embodiments, the detector barcodes are cleaved from the corresponding detector molecules by being subject to a trigger. In some embodiments, as described herein, the trigger comprises a deconstructor molecule, a trigger signal, or combinations thereof. In some embodiments, the deconstructor molecule comprises a detector barcode release molecule (e.g., reference character 29 from FIGS. 4 and 7).

In some embodiments, the cleaved detector barcodes 21 are isolated (reference character 108 FIG. 12) from the solution comprising the supramolecular structures. In some embodiments, the cleaved detector barcodes 21 are isolated from the solution through polyethylene glycol (PEG) precipitation. In some embodiments, the cleaved detector barcodes 21 are isolated from the solution by binding the core structures in the solution to microbeads, solid support and/or magnetic beads through a corresponding anchor molecule on the respective core structure, followed by isolation of the cleaved detector barcodes through centrifugation, micron filtration, chromatography or combinations thereof.

In some embodiments, the cleaved detector barcodes provide a signal that correlates to the respective analyte molecule bound to the respective detector molecule. In some embodiments, as described herein, the detector barcode comprises a DNA strand. In some embodiments, the detector barcode provides a DNA signal correlating to the analyte molecule. In some embodiments, as depicted in FIG. 12 reference character 110, the isolated detector barcodes 21 are analyzed to identify and/or quantify the corresponding analyte molecules in the sample. In some embodiments the analysis of the isolated detector barcodes comprises genotyping, qPCR, sequencing, or combinations thereof.

In some embodiments, the method for detecting analyte molecules as depicted in FIG. 12 comprises cleaving the capture barcode 20 from a corresponding capture molecules that are linked to a respective detector molecule (e.g., as located on a supramolecular structure that shifted to a stable state). In some embodiments, the capture barcodes 20 are cleaved from the corresponding detector molecules through nucleic acid (DNA/RNA) strand displacement, optical cleavage, chemical cleavage, or a combination thereof. In some embodiments, the detector barcodes are cleaved from the corresponding detector molecules by being subject to a trigger. In some embodiments, as described herein, the trigger comprises a deconstructor molecule, a trigger signal, or combinations thereof. In some embodiments, the deconstructor molecule comprises a capture barcode release molecule (e.g., reference character 31 from FIGS. 4 and 7).

In some embodiments, the cleaved capture barcodes 20 are isolated (reference character 108 FIG. 12) from the solution comprising the supramolecular structures. In some embodiments, the cleaved capture barcodes 20 are isolated from the solution through polyethylene glycol (PEG) precipitation. In some embodiments, the cleaved capture barcodes 20 are isolated from the solution by binding the core structures in the solution to microbeads, solid support and/or magnetic beads through a corresponding anchor molecule on the respective core structure, followed by isolation of the cleaved capture barcodes through centrifugation, micron filtration, chromatography or combinations thereof.

In some embodiments, the cleaved capture barcodes provide a signal that correlates to the respective analyte molecule bound to the respective detector molecule. In some embodiments, as described herein, the capture barcode comprises a DNA strand. In some embodiments, the capture barcode provides a DNA signal correlating to the analyte molecule. In some embodiments, as depicted in FIG. 12 reference character 110, the isolated capture barcodes 21 are analyzed to identify and/or quantify the corresponding analyte molecules in the sample. In some embodiments the analysis of the isolated capture barcodes comprises genotyping, qPCR, sequencing, or combinations thereof.

The preceding relates example implementations and variations in which analysis and isolation is primarily performed after isolation and/or removal of unbound detector molecules (i.e., detector molecules to which molecule(s) of analyte have bound) such that the bound detector molecules form the basis for the quantitative and/or qualitative detection process. However, it may be appreciated by those skilled in the art that corresponding information, under the principle of the preservation of complementary knowledge, may be determined in such implementations, as well as other embodiments discussed herein, by instead isolating and/or removing the bound detector molecules such that the analysis is performed on the unbound detector molecules. That is, knowledge of the full set of capture and detector molecules employed allows detection of either the bound or the unbound detector molecules to be correlated to a qualitative or quantitative assessment of one or more analytes of interest.

Supramolecular Structures Provided with Hydrogel Beads or Solid Substrate

As described herein, in some embodiments, one or more supramolecular structures are provided with one or more hydrogel beads and/or more one or more solid substrates. In some embodiments, the hydrogel bead comprises one or more supramolecular structures polymerized to a hydrogel matrix. FIG. 13 provides an exemplary embodiment for forming a hydrogel bead 120, wherein in addition to combining one or more monomers 122 and one or more crosslinking molecules 124 to form a hydrogel, one or more supramolecular structures 40 are introduced. In some embodiments, the one or more supramolecular structures 40 co-polymerizes with the hydrogel matrix, forming the hydrogel bead 120. In some embodiments, hydrogel bead 120 comprises the one or more supramolecular structures attached to the hydrogel matrix. In some embodiments, the hydrogel bead 120 comprises the one or more supramolecular structures embedded within the hydrogel matrix. In some embodiments, each respective anchor molecule 18 of the one or more supramolecular structures 40 co-polymerizes with the hydrogel matrix 120. In some embodiments, the one or more monomers 122 comprise an acrylamide. In some embodiments, the one or more cross-linkers comprise a bis-acrylamide. In some embodiments, each hydrogel bead is formed using microfabrication tools. In some embodiments, each hydrogel bead is formed using emulsion polymerization. FIG. 14 provides an exemplary embodiment for forming a hydrogel bead 120, which comprises trapping 126 one or more monomers, one or more crosslinkers, and one or more supramolecular structures 40 within a droplet. In some embodiments, the droplet is an oil droplet. In some embodiments, the droplet dimension are specified. In some embodiments, polymerization occurs within the droplet thereby forming the one or more hydrogel beads 120. In some embodiments, polymerization occurs through interaction with an initiator and/or catalyst.

FIG. 15 provides an exemplary embodiment wherein one or more supramolecular structures 40 are attached to a solid substrate 128. In some embodiments, each anchor molecule 18 of a supramolecular structure 40 links with the solid surface of the solid substrate 128. In some embodiments, the solid substrate 128 comprises a microparticle. In some embodiments, the microparticle comprises a polystyrene particle, silica particle, magnetic particle or paramagnetic particle. In some embodiments, the solid substrate 128 comprises a microbead. In some embodiments, the microbead comprises a polystyrene bead, silica bead, magnetic bead or paramagnetic bead.

As described herein, in some embodiments, a plurality of supramolecular structures embedded within a single hydrogel bead or attached to a solid substrate are spaced apart with a prescribed distance so as to limit or eliminate cross-reactivity (cross-talk, intermolecular interaction) with other supramolecular structures. In some embodiments, the number, size, and/or stoichiometry of the supramolecular structures attached to each hydrogel bead or solid substrate are specified so as to achieve a prescribed distance between each supramolecular structure. In some embodiments, the surface and volumetric density of the supramolecular structures attached to each hydrogel bead or solid substrate are controlled to minimize or eliminate intermolecular interactions and thereby reducing the possibility of cross-talk between the plurality of supramolecular structures. In some embodiments, the distance between any two supramolecular structures on a given hydrogel bead or solid substrate (e.g., microparticle) is larger than a maximum distance between capture and detector molecules of a supramolecular structure, so as to minimize intermolecular interactions between molecules from different supramolecular structures.

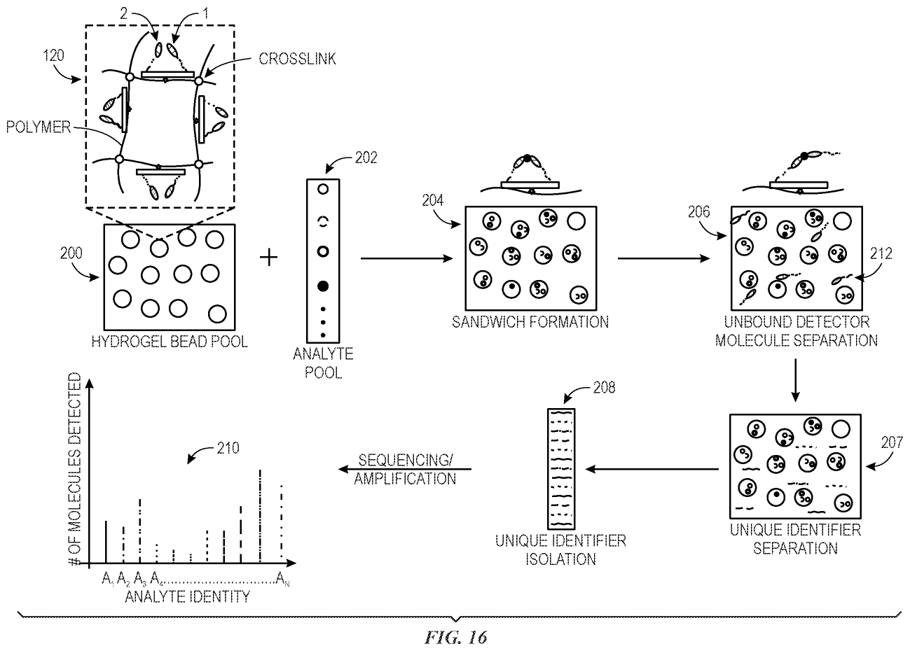

FIG. 16 provides an exemplary method for detecting one or more analyte molecules in a sample using one or more supramolecular structures embedded within one or more hydrogel beads or attached to one or more solid substrates (e.g., microparticles). FIG. 16 depicts an exemplary embodiment where a hydrogel bead pool 200 is provided, wherein one or more supramolecular structures are embedded within one or more hydrogel beads 120. In some embodiments, alternate to a hydrogel bead pool, a solid substrate pool is provided, wherein one or more supramolecular structures are attached to one or more solid substrates (e.g., microparticle), as described herein and shown in FIG. 15. In some embodiments, the sample, comprising one or more analyte molecules (e.g., analyte pool 202) is contacted with the supramolecular structures embedded within the hydrogel beads 120. In some embodiments, the sample comprises an aqueous solution, and is mixed with the hydrogel bead pool 200 to form a combined solution. In some embodiments, contacting the sample with the supramolecular structures comprises incubating the sample with the supramolecular structures. In some embodiments, the sample and supramolecular structures are incubated in an incubator with prescribed environmental conditions. In some embodiments, the sample is incubated with the supramolecular structures for a time period from about 30 seconds to about 24 hours. In some embodiments, the sample is incubated with the supramolecular structures for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

With continued reference to FIG. 16, in some embodiments, the supramolecular structures are all in an unstable state (as shown with the exemplary hydrogel bead 120). In some embodiments, and as described herein, interaction between an analyte molecule and corresponding capture 2 and detector 1 molecules shift the respective supramolecular structure from the unstable state to a stable state (e.g., a sandwich formation with the capture molecule, analyte molecule, and detector molecule as shown with reference character 204). In some embodiments, a particular type of analyte molecule will bind with a particular pair of capture and detector molecules. In some embodiments, a given pair of capture and detector molecules are configured to bind with more than one type of analyte molecule. In some embodiments, the switching from an unstable state to a stable state for any given supramolecular structure is dependent on the specific capture and detector molecules bound thereto and the analyte molecules in the sample. In some embodiments, given that the state-change of the supramolecular structure is primarily dependent on the intra-molecular interactions (on the supramolecular nanostructure), potential intermolecular interactions between two different supramolecular structures are minimized or eliminated by limiting the net concentration of the supramolecular structures embedded within a hydrogel bead or attached to a solid substrate (as described herein), such that the mean distance between any two supramolecular structures is larger than maximum intramolecular distance between a pair of capture and detector molecules on a given supramolecular structure.

In some embodiments, after contacting the sample, at least one of the supramolecular structures moves to a stable state (e.g., sandwich formation wherein the capture molecule, analyte molecule and detector molecule are simultaneously linked together), while at least one of the supramolecular structures remains in an unstable state as the respective capture and detector molecules did not bind or interact with an analyte molecule from the sample.

With continued reference to FIG. 16, after the sample has been contacted with the supramolecular structures for a prescribed amount of time, the combined solution of the sample and supramolecular structures is subjected to a trigger so as to cleave a linkage between the detector molecule and the core structure for the respective supramolecular structures. In some embodiments, the trigger comprises introducing a solution comprising one or more deconstructor molecules (e.g., detector deconstructor molecule, reference character 28 from FIGS. 4,7) to the combined solution. In some embodiments, the trigger comprises subjecting the combined solution to a trigger signal. In some embodiments, the trigger comprises a combination of introducing a deconstructor molecule in the combined solution and subjecting the combined solution to a trigger signal. In some embodiments, as described herein, the deconstructor molecule comprises a nucleic acid (DNA or RNA), a peptide, a small organic molecule, or combinations thereof. In some embodiments, as described herein, the trigger signal comprises an electrical signal, microwave signal, ultraviolet illumination, visible illumination or near infra-red illumination. In some embodiments, the combined solution is subjected to the trigger for a prescribed amount of time. In some embodiments, the combined solution is incubated with one or more deconstructor molecules for a prescribed amount of time. In some embodiments, the combined solution is incubated with the deconstructor molecules for a time period from about 30 seconds to about 24 hours. In some embodiments, the combined solution is incubated with the deconstructor molecules for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

As shown in FIG. 16, reference character 206, in some embodiments, subjecting the combined solution to the trigger cleaves a linkage between a detector molecule and respective core structure, such as through the linkage between a detector barcode (e.g., reference character 21 FIG. 1) and the core structure 13. In some embodiments, the cleavage is achieved through nucleic acid (DNA/RNA) strand displacement, optical cleavage, chemical cleavage, another technique known in the art, or combinations thereof. For supramolecular structures that moved to a stable state, the detector molecule 1 is shown as remaining to be linked to the core structure 13 via linkage with the corresponding capture molecule 2. For supramolecular structures that remained in an unstable state, the detector molecule is shown as being unbound 212 from the respective core structure. In some embodiments, the unbound detector molecules are separated from the hydrogel bead (or solid substrate), as shown with reference character 206. In some embodiments, the unbound detector molecules 2 remain linked to the respective detector barcodes 21.

In some embodiments, the unbound detector molecules and corresponding detector barcodes are further separated from the combined solution. In some embodiments, the unbound detector molecules are separated from the combined solution through polyethylene glycol (PEG) precipitation. In some embodiments, the unbound detector molecules are separated from the combined solution by binding the core structures in the combined solution to microbeads, solid support and/or magnetic beads through a corresponding anchor molecule on the respective core structure, followed by separation of the unbound detector molecules through centrifugation, micron filtration, chromatography or combinations thereof.

In some embodiments, after the unbound detector molecules have been separated from the combined solution, the detector barcodes 21 are cleaved from the corresponding detector molecules linked to a respective capture molecule (e.g., as located on a supramolecular structure that shifted to a stable state). In some embodiments, the detector barcodes are cleaved from the corresponding detector molecules through nucleic acid (DNA/RNA) strand displacement, optical cleavage, chemical cleavage, or a combination thereof. In some embodiments, the detector barcodes are cleaved from the corresponding detector molecules by being subject to a trigger. In some embodiments, as described herein, the trigger comprises a deconstructor molecule, a trigger signal, or combinations thereof. In some embodiments, the deconstructor molecule comprises a detector barcode release molecule (e.g., reference character 29 from FIGS. 4 and 7).

As shown with reference character 207 in FIG. 16, in some embodiments, the cleaved detector barcodes 21 are separated from the corresponding hydrogel bead or solid substrate. In some embodiments, the cleaved detector barcodes 21 are isolated (reference character 208 FIG. 16) from the solution comprising the supramolecular structures. In some embodiments, the cleaved detector barcodes 21 are isolated from the solution through polyethylene glycol (PEG) precipitation. In some embodiments, the cleaved detector barcodes 21 are isolated from the solution by binding the core structures in the solution to microbeads, solid support and/or magnetic beads through a corresponding anchor molecule on the respective core structure, followed by isolation of the cleaved detector barcodes through centrifugation, micron filtration, chromatography or combinations thereof.

In some embodiments, the cleaved detector barcodes 21 provide a signal that correlates to the analyte molecule bound to the respective detector molecule. In some embodiments, as described herein, the detector barcode comprises a DNA strand. In some embodiments, the detector barcode provides the DNA signal correlating to the analyte molecule. In some embodiments, as depicted in FIG. 16 reference character 210, the isolated detector barcodes 21 are analyzed to identify the corresponding analyte in the sample. In some embodiments, the isolated detector barcodes 21 are analyzed to identify and/or quantify the corresponding analyte molecules in the sample. In some embodiments the analysis of the isolated detector barcodes comprises genotyping, qPCR, sequencing, or combinations thereof.

Detecting Analyte Molecules within a Single Cell

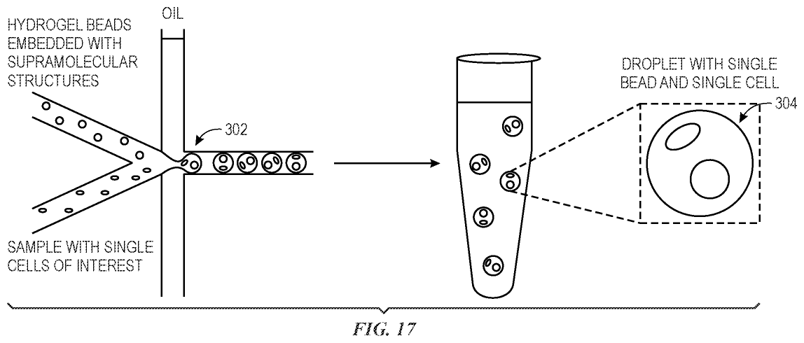

FIGS. 17-20 provide an exemplary method for detecting analyte molecules located within a single cell. In some embodiments, attaching supramolecular structures to a hydrogel bead, or attaching supramolecular structures onto a solid substrate (e.g., microbeads), enables the detection of intracellular analyte molecules (e.g., protein, antigen) and quantification of intracellular analyte molecules (e.g., protein, antigen) at single cell resolution. In some embodiments, the intracellular analyte molecules may not exist outside the respective cell. In some embodiments, the detection of intracellular analyte molecules (e.g., protein, antigen) and quantification of intracellular analyte molecules (e.g., protein, antigen) at single cell resolution comprises a single-cell proteomics assay. FIGS. 17-20 provide an exemplary method for detecting analyte molecules wherein the supramolecular structures are provided as embedded within hydrogel beads. In some embodiments, the method depicted in FIGS. 17-20 alternatively comprises providing the supramolecular structures as attached to solid substrates (e.g., microbeads).

FIG. 17 provides an exemplary first step comprising using a microfluidic droplet formation chip to trap 302 single cells with hydrogel beads that are attached with one or more supramolecular structures, wherein each droplet 304 formed encloses a single cell and hydrogel bead. In some embodiments, each droplet 304 encloses one or more single cells and one or more hydrogel beads. In some embodiments, the supramolecular structures are attached to the hydrogel beads (e.g., embedded within the hydrogel beads) using methods as described herein (e.g., FIGS. 13-14). In some embodiments, the one or more supramolecular structures are configured to interact with specific intercellular analyte molecules (e.g., proteins, antigens). In some embodiments, other methods and/or microfluidic chip designs are used to achieve the trapping of single cells with the one or more hydrogel beads.

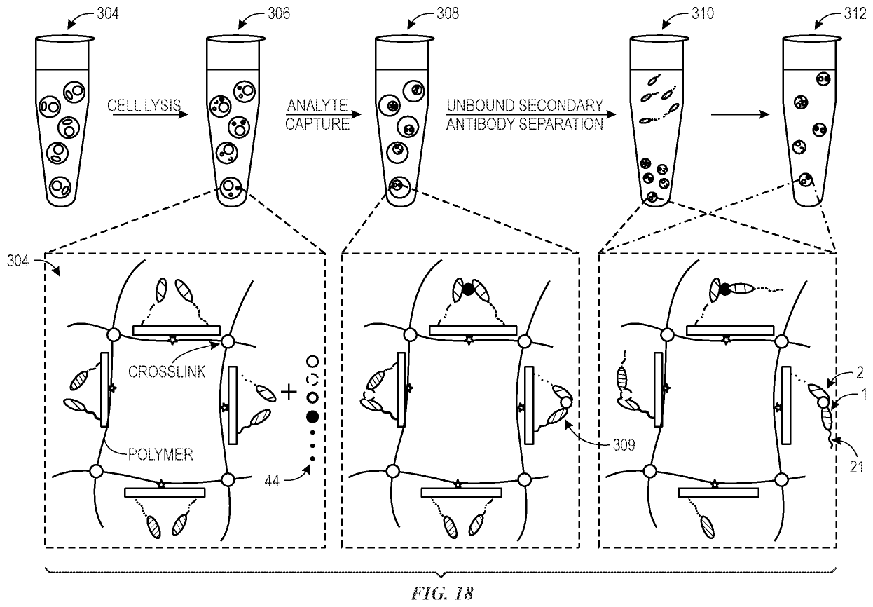

FIG. 18 provides an exemplary embodiment for collecting the droplets 304, having the trapped single cells and hydrogel beads, in a combined solution, and processing the droplets 304. In some embodiments, the intracellular analyte molecules (e.g., proteins, antigens) are transferred from each cell onto or about the hydrogel bead within the same droplet 304. In some embodiments, transferring the intracellular analyte molecules comprises lysing the cell that is trapped in the droplet (reference character 306 in FIG. 18, step 1). In some embodiments, lysing comprises mechanical processing or introducing a lysis buffer. As depicted in the combined solution, in some embodiments, all the supramolecular structures corresponding to a hydrogel bead is in an unstable state. In some embodiments, the contents of the lysate (e.g., analyte molecules 44) is subsequently allowed to interact 308 (step 2) with the hydrogel bead within the droplet, thereby enabling specific intercellular analyte molecules (e.g., proteins, antigens) to be captured by associated supramolecular structures attached with the hydrogel bead (e.g., capture 2 and detector 1 molecules). In some embodiments, the contents of the lysate (e.g., analyte molecules) is allowed to interact with the hydrogel bead for a time period from about 30 seconds to about 24 hours. In some embodiments, the contents of the lysate (e.g., analyte molecules) is allowed to interact with the hydrogel bead for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

In some embodiments, and as described herein, interaction between an analyte molecule and corresponding capture 2 and detector 1 molecules shifts the respective supramolecular structure from the unstable state to a stable state (as shown with reference character 309). In some embodiments, a particular type of analyte molecule will bind with a particular pair of capture and detector molecules (e.g., a sandwich formation with the capture molecule, analyte molecule, and detector molecule). In some embodiments, a given pair of capture and detector molecules are configured to bind with more than one type of analyte molecule. In some embodiments, the switching from an unstable state to a stable state for any given supramolecular structure is dependent on the specific capture and detector molecules bound thereto and the analyte molecules in the cell.

After the contents of the lysate have been allowed to interact with the hydrogel for a prescribed amount of time, in some embodiments, the droplets are subsequently broken, after which the hydrogel beads are washed. In some embodiments, the hydrogel beads are subjected to a trigger so as to cleave a linkage between the detector molecule and the core structure for the respective supramolecular structures. In some embodiments, the trigger comprises introducing a solution comprising one or more deconstructor molecules (e.g., detector deconstructor molecule 28 from FIGS. 4,7) to the combined solution comprising the hydrogel beads (reference character 310). In some embodiments, the trigger comprises subjecting the combined solution to a trigger signal. In some embodiments, the trigger comprises subjecting the combined solution to a deconstructor molecule and a trigger signal. In some embodiments, as described herein, the deconstructor molecule comprises a nucleic acid (DNA or RNA), a peptide, a small organic molecule, or combinations thereof. In some embodiments, as described herein, the trigger signal comprises an electrical signal, microwave signal, ultraviolet illumination, visible illumination or near infra-red illumination. In some embodiments, the hydrogel beads are subjected to the trigger for a prescribed amount of time. In some embodiments, the hydrogel beads are subjected to the trigger from about 30 seconds to about 24 hours. In some embodiments, the hydrogel beads are subjected to the trigger from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

In some embodiments, the trigger (e.g., detector deconstructor molecule 28 from FIGS. 4,7) releases all the detector molecules from the hydrogel beads that are not linked to a corresponding capture molecule (i.e., not participating in the sandwich formation comprising a capture molecule, detector molecule, and analyte molecule).

In some embodiments, after the hydrogel beads have been subjected to the trigger for a prescribed amount of time, the hydrogel beads are washed one or more times to remove any weakly bound analyte molecules (e.g., proteins, antigens) or any detector molecules unbound from a respective supramolecular structure (reference character 312, step 4). In some embodiments, after being washed a prescribed number of times, each hydrogel bead contains analyte molecules (e.g., protein, antigens) that have been specifically captured from a single cell.

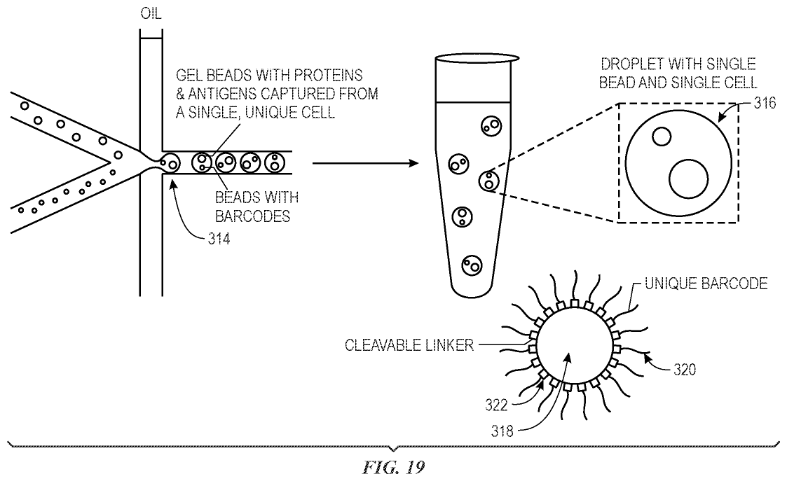

FIGS. 19-20 provide an exemplary depiction of a method for analyzing the content of each resulting hydrogel bead from the method depicted in FIG. 18. In some embodiments, the content of each hydrogel bead is analyzed independently, wherein each hydrogel bead is barcoded individually. FIG. 19 provides an exemplary illustration of a microfluidic droplet formation system that is designed to form droplets 316 that enclose 314 1) a single hydrogel bead, that is carrying within itself one or more analyte molecules (e.g., protein, antigen) from a single cell, with 2) a unique barcode bead 318. In some embodiments, each barcode bead comprises a unique nucleic acid strand 320 that is between 20 and 60 bases long and connected to the bead through a linker 322 that can be cleaved. In some embodiments, the cleavable linker on the barcode bead is broken using an electromagnetic signal (light) or a chemical signal.

FIG. 20 provides an exemplary illustration of a method for transferring the unique barcode onto each hydrogel bead, both of which are present in a single droplet 316 (reference character 324, step 1). In some embodiments, the barcode 320 is cleaved from the barcoding beads 318 and allowed to interact with the hydrogel beads in the respective droplet 316. As described herein, the barcode 320 is cleaved from the barcoding bead 318 by being subjected to an electromagnetic signal (e.g., light, UV light, DTT) or chemical signal. In some embodiments, cleaving the barcode 320 from the barcoding bead leads to the barcode 320 binding to a detector barcode 21 on a supramolecular structure within the respective droplet 316 (reference character 326, step 2). In some embodiments, the droplets are subsequently broken. In some embodiments, barcode strands 320 that did not bind with a detector barcode are separated (reference character 328). In some embodiments, the hydrogel beads are washed to remove any remaining barcoding strands from the solution (reference character 320). In some embodiments, the barcoded detector barcodes 332 are separated from the detector molecules and further analyzed. In some embodiments, the barcoded detector barcodes 332 are cleaved from the corresponding detector molecules through nucleic acid (DNA/RNA) strand displacement, optical cleavage, chemical cleavage, or a combination thereof. In some embodiments, the detector barcodes are cleaved from the corresponding detector molecules by being subject to a trigger. In some embodiments, as described herein, the trigger comprises a deconstructor molecule, a trigger signal, or combinations thereof.

In some embodiments, each separated barcoded detector barcode 332 will have two sections: a first section 320 that has the unique barcode 320 that identifies a unique cell, and a second section 21 that provides the identity of the analyte molecule (e.g., protein or antigen). In some embodiments, taken together, the analysis of the barcoded detector barcode 332 enables the concentration of intracellular analyte molecules (e.g., protein, antigen) to be profiled at single cell resolution. In some embodiments, the barcoded detector barcodes 332 are analyzed to identify and/or quantify the corresponding analyte molecules in the sample. In some embodiments the analysis of the barcoded detector barcodes 332 comprises genotyping, qPCR, sequencing, or combinations thereof.

### Detection of Analyte Molecules Using a Surface Assay

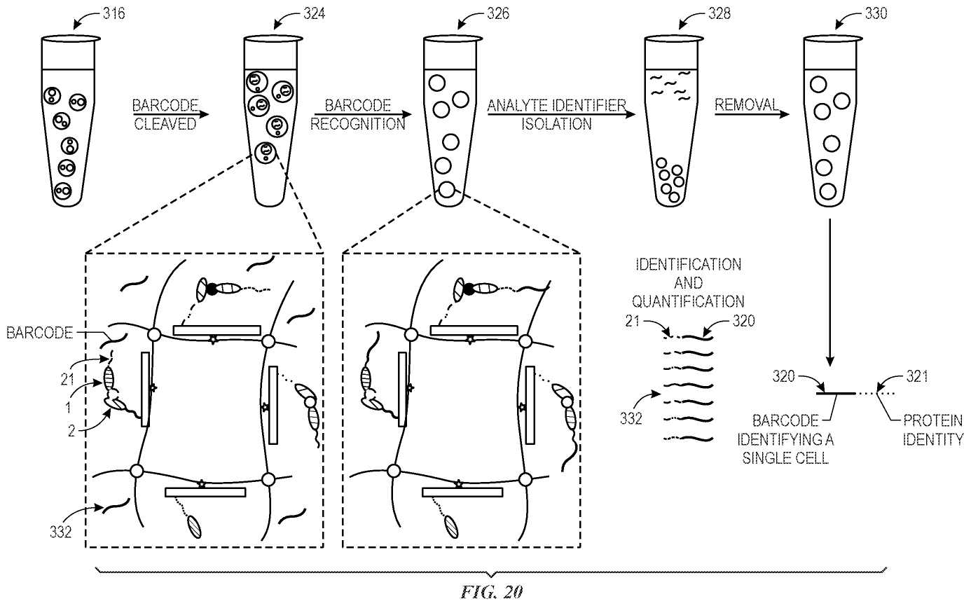

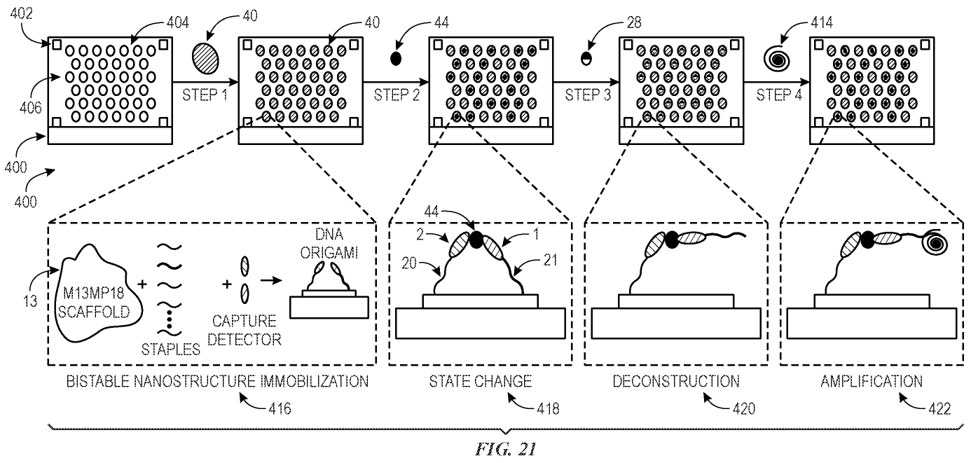

FIG. 21 provides an exemplary illustration of a method for detecting analyte molecules in a sample using a surface based assay that uses supramolecular structures, as described herein, for single-molecule counting of analytes in the sample (i.e., detecting analyte molecules in the sample at a single molecule resolution). In some embodiments, the supramolecular structures comprise a core structure comprising a DNA origami core. In some embodiments, a substrate 400 is provided that may be of various configurations, including having a surface or surfaces that is entirely planar, partially planar (e.g., having localized surface features or elements that are planar), entirely curved (e.g., a tube or cylinder), or partially curved (e.g., having localized surface features or elements that are planar). While certain embodiments may provide a uniform or consistent chemical environment across the substrate surface corresponding to an active area, in other embodiments localized, differential chemical environments may be provided on the surface of the substrate in either random or ordered arrangements (e.g., differential chemical patterning or etching, localized surface and/or chemical features corresponding to different chemical patterning or environments, and so forth). Example of localized surface features that may be associated with different chemical patterning, coating, or environments include, but are not limited to, trenches, platforms, pedestals, wells, and so forth. In certain embodiments the substrate 400 may comprise (a) Fiduciary markers 402 that serve as a reference coordinates for all the features on the substrate 400; (b) A defined set of micropatterned binding sites 406 where individual core structures (e.g., DNA origami) may be immobilized; (c) background passivation 404 that minimizes or prevents interaction between the surface of the substrate 400 and the supramolecular structure (including capture and detector molecules, core structure molecules). In some embodiments, the fiduciary markers comprise geometric features defined on a surface to be used as reference features for other features on the substrate. In some embodiments, the fiduciary markers 402 are coated with a polymer or self-assembled monolayer that does not interact with a core structure or other molecules of the supramolecular structure (e.g., DNA origami). In some embodiments, the background passivation 404 minimizes or prevents interaction between the surface of the substrate 400 and analyte molecules of the sample. In some embodiments, the substrate 400 comprises optical or electrical devices like FET, ring resonators, photonic crystals or microelectrode, to be defined prior to the formation of the binding sites 406. In some embodiments, the binding sites 406 are micropatterned on the substrate 400. In some embodiments, the binding sites 406 on the surface are in a periodic pattern. In some embodiments, the binding sites 406 on the surface are in a non-periodic pattern (e.g., random). In some embodiments, a minimum distance is specified between any two binding sites 406. In some embodiments, the minimum distance between any two binding sites 406 is at least about 200 nm. In some embodiments, the minimum distance between any two binding sites 406 is from at least about 40 nm to about 5000 nm. Based on substrate considerations, in other implementations the minimum distance between any two binding sites 406 may even be within the range from at least about 5 μm to about 100 μm. In some embodiments, the geometric shape of the binding sites 406 comprises a circle, square, triangle or other polygon shapes. In some embodiments, the chemical groups that are used for passivation 404 comprise neutrally charged molecules like a Tri-methyl silyl (TMS), an uncharged polymer like PEG a zwitterionic polymer like, or combinations thereof. In some embodiments, the chemical group used to define the binding site 406 comprises a silanol group, carboxyl group, thiol, other groups, or combinations thereof.

In some embodiments, a single supramolecular structure 40 is attached to a respective binding site 406 (Step 1). Reference character 416 provides a depiction of the components of the supramolecular structure 40, individually and as assembled and arranged on the substrate (components are as described herein, e.g., FIGS. 1, 2-3, 5-6). In some embodiments, the supramolecular structure 40 comprises a core structure 13 comprising a DNA origami, wherein the supramolecular structures 40 is attached onto each of the binding sites using DNA origami placement technique (step 1). In some embodiments, the supramolecular structure 40 is assembled prior to being attached to a respective binding site 406. In some embodiments, the DNA origami comprises a unique shape and dimension, so as to facilitate binding to a binding site using the DNA origami placement technique. In some embodiments, DNA origami placement comprises a directed self-assembly technique for organizing individual DNA origami (e.g., a core structure) on a surface (e.g., micropatterned surface). In some embodiments, alternatively to the DNA origami placement, a reactive group of the supramolecular nanostructure 40 is bound to a DNA origami that has been pre-organized on the binding site. In some embodiments, both of these methods for binding a supramolecular nanostructure to a corresponding binding site rely on the ability to organize one or more molecules on a micropatterned binding site using the DNA origami placement technique. In some embodiments, the substrate could be stored for a significant period after this step, in a clean environment.

With continued reference to FIG. 21, in some embodiments, a sample (as described herein) comprising analyte molecules is contacted with the substrate (step 2). In some embodiments, the sample is contacted with the substrate using a flow-cell. In some embodiments, the sample is incubated on the substrate with the supramolecular structures attached to the binding sites 406. In some embodiments, the incubation period may be from about 1 second or less to about 48 hours. By way of example, and to provide illustrative, but non-limiting example incubation period ranges, in some embodiments the incubation period may be from about 1 second (or less) to about 1 minute, from about 1 second (or less) to 30 seconds, from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours. Further, depending on the sensing or read-out mechanism employed, response times (which may correspond to incubation period in such contexts) may be in real-time (e.g., less than 1 second). For example, use of field effect transistors or other electrical read-out sensing mechanisms may allow responses to be measured in real-time.

In some embodiments, the analyte molecules 44, in the sample, interact with the supramolecular structures 40 on the surface 400. In some embodiments, a single copy of a specific analyte molecule 44 binds simultaneously with both the capture and detector molecules, such that the particular supramolecular structure switches from an unstable state to a stable state 418 (as described herein, e.g., FIGS. 8-10). In some embodiments, a single copy of a particular analyte might interact simultaneously with the capture and detector molecules that are already bound to each other and switch the supramolecular structure from a stable state to an unstable state (as described herein, e.g., FIG. 11).

With continued reference to FIG. 21, in some embodiments, the substrate is then subjected to a trigger. In some embodiments, the trigger comprises a deconstructor molecule (e.g., detector deconstructor molecule 28 in FIG. 7). In some embodiments, the trigger comprises a trigger signal. In some embodiments, as described herein, the deconstructor molecule (e.g., detector deconstructor molecule 28) comprises a nucleic acid (DNA or RNA), a peptide, a small organic molecule, or combinations thereof. In some embodiments, as described herein, the trigger signal comprises an electrical signal, microwave signal, ultraviolet illumination, visible illumination or near infra-red illumination. In some embodiments, deconstructor molecules associated with the supramolecular structures attached to the substrate is allowed to interact with said supramolecular structures. In some embodiments, the deconstructor molecules are introduced into the flow-cell containing the substrate. In some embodiments, the deconstructor molecule is incubated with the supramolecular structures from about 30 seconds to about 24 hours (step 3). In some embodiments, the incubation period may be from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

In some embodiments, interaction with the deconstructor molecule cleaves the detector molecules and detector barcodes of all the supramolecular structures in the unstable state, such that these detector molecules and detector barcodes will be physically cleaved from the substrate 400. In some embodiments, the physically cleaved detector molecules and detector barcodes are removed during one or more buffer washes at the end of the incubation step. In some embodiments, wherein supramolecular structures on the substrate had shifted to a stable state, due to the capture of single analyte molecules, the corresponding detector molecules and detector barcodes are still linked to the supramolecular structure 420, and thereby stably bound to the substrate due to the analyte mediated sandwich formed between the corresponding detector and capture molecules (i.e., linkage between the capture molecule, analyte molecule, and detector molecule).

With continued reference to FIG. 21, in some embodiments, the detector barcode at the location of supramolecular structure that shifted to a stable state is used as a binding site 422 for a signaling element 414 (step 4). In some embodiments, the signaling element comprises a fluorescent molecule or microbead, a fluorescent polymer, highly charged nanoparticles or polymer. In some embodiments, one or more signaling elements are allowed to interact with the supramolecular structures on the structure. In some embodiments, the signaling elements are introduced into the flow-cell containing the substrates. In some embodiments, the detector barcode is used as a polymerization initiator for growth of highly fluorescent polymer in a process such as rolling circle amplification or hybridization chain reaction, bridge amplification, or, exclusion amplification based approaches.

In some embodiments, introduction of the signaling element 414 as described with step 4 leads to a surface in which every individual analyte capture event (i.e. linkage between the capture molecule, detector molecule, and analyte molecule) leads to a signaling element being present at the location of the respective analyte (as linked with the capture and detector molecules). In some embodiments, the signaling element is optically active and can be measured using a microscope or integrated optical sensor within the substrate 400. In some embodiments, the signaling element is electrically active and may be measured using an integrated electrical sensor. In some embodiments, the signaling element is magnetically active and may be measured using an integrated magnetic sensor. In some embodiments, each signal event is associated with the capture of the same type of analyte molecule (a single copy of the same type of analyte molecule), determined by the corresponding detector and capture molecule, thus counting the number of locations where the signaling element is present gives the quantification of the concentration of the analyte molecule in the sample.

In some embodiments, the method for detecting an analyte as described in FIG. 21 uses a supramolecular core wherein the core structure is bound to a DNA origami already organized on the surface of the substrate through a respective anchor moiety of the core structure.

In some embodiments, the method for detecting an analyte as described in FIG. 21 enables the detection of a single type of analyte molecule. In some embodiments, the method for detecting an analyte as described in FIG. 21 enables detection of a plurality of types of analyte molecules (multiplexed analyte molecule detection). In some embodiments, each supramolecular structure is barcoded to uniquely identify the respective capture and detector molecules associated with the supramolecular structure, thereby enabling the respective analyte molecule captured to be identified based on the barcode(s). In some embodiments, each supramolecular structure is barcoded using the respective anchor molecule. As discussed herein, the capture and detector molecules may be antibodies, nanobodies, aptamers, Somamers, oligonucleotides, or small molecules that have an affinity to the analytes being probed.

### Detection of Analyte Molecules Using a Microfluidic Structure—Example Implementations

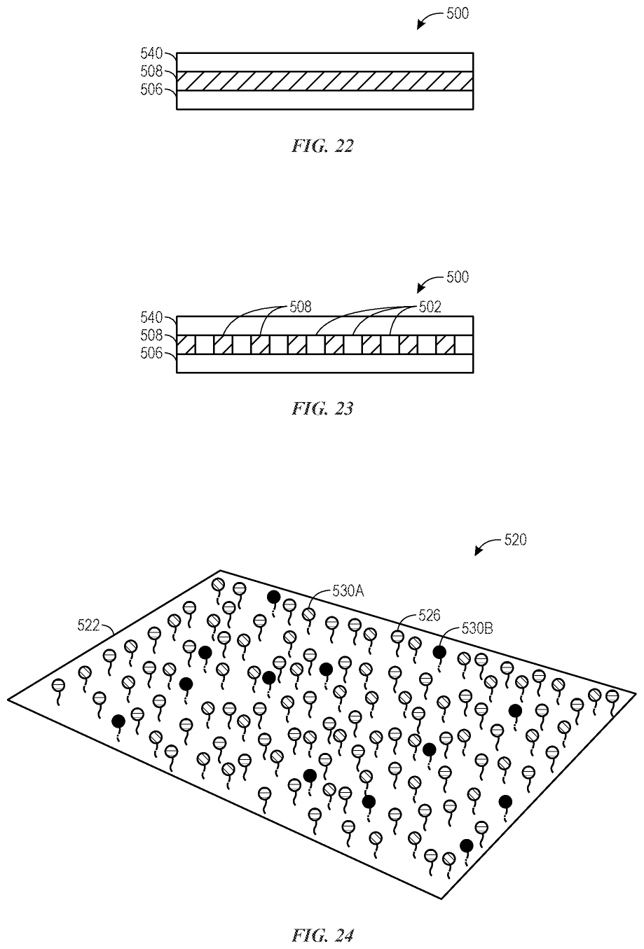

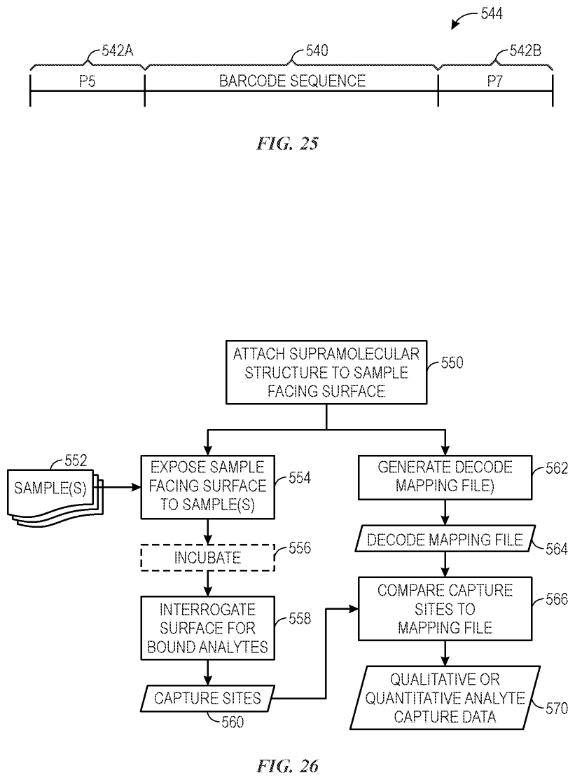

With the preceding discussion of analyte detection using a surface assay in mind, further examples are provided related to use of a microfluidic device 500 as a substrate for attachment of the supramolecular structure 40. In these examples, a microfluidic device 500 may be provided having a substrate on which supramolecular structures 40 as described herein may bind, either in random arrangements or at ordered or patterned locations that are chemically adapted for binding the supramolecular structures 40. In practice, a microfluidic device 500 as used herein may be understood to include entry and exit lanes through which a fluid (e.g., a fluid sample) may be flowed. With reference to FIGS. 22 and 23, such a device may be formed by a top substrate 504 and bottom substrate 506 (defining the upper and lower bounds of the fluid passages) with an interposer 508 spacing apart the top substrate 504 and bottom substrate 506 and defining the sidewalls and geometry of the microfluidic passages 502 and sample chambers.

As discussed herein, a substrate surface of the microfluidic device 500 over which a fluid sample may be flowed is provided and used as an attachment surface for supramolecular structures 40, or affinity binders (e.g., antibodies, aptamers, nanobodies, and so forth) cleaved from such a supramolecular structure 40, which are capable of binding molecules of interest (e.g., DNA, RNA, protein, peptides, metabolites, or any biologically relevant molecule). As discussed herein, the binding mechanism of the supramolecular structure 40 for the molecules of interest (e.g., analyte 44) may be specific to a particular molecule (e.g., protein) of interest so as to attach to and capture the molecule of interest with specificity. While in the simplest scenario a given microfluidic structure 500 may be configured to include a single probe type (corresponding to a single molecule of interest). At the other bound, a given microfluidic structure 500 may instead be configured to include tens, hundreds, thousands, millions, or billions of probe types, each corresponding to different molecules of interest.

Turning to FIG. 24, a two-dimensional (2D), sample-facing surface 520 of a microfluidic sample chamber or passage is illustrated in accordance with the present examples. In one such implementation, the sample-facing surface 520 is coated in a hydrogel or other suitable coating 522 that includes one or more universal adapters or attachment mechanisms. By way of example, the surface 520 may be seeded with or have chemically attached a corresponding (e.g., complementary or otherwise chemically hybridizing) attachment molecule 526 capable of forming an attachment to an anchor molecule 18, as described herein. As may be appreciated, attachment molecules 526 may distributed randomly on the surface 520 (such that supramolecular structures 40 attach in a randomized or undirected manner to the surface 520) or may be distributed in an ordered or patterned manner (such that supramolecular structures 40 attach in an ordered or otherwise constrained manner to the surface 520). By way of illustrative and non-limiting example, the attachment molecule 526 may be a suitable oligomer, biotin, streptavidin, and so forth, such as an oligonucleotide that is complementary to a portion of the anchor molecule 18 of a supramolecular structure 44, such that interaction between the attachment molecule 526 and complementary anchor molecule 18 binds the supramolecular structure 40 to the surface 520. More generally, the anchor molecule 18 may comprise a reactive molecule and thus the attachment molecule 526 may correspondingly comprise a molecule with which the anchor molecule 18 reacts. In some embodiments the anchor molecule 18 comprises a DNA strand capable of interacting (e.g., hybridizing) with a complementary nucleic acid strand as the attachment molecule 526. In further embodiments, the anchor molecule 18 comprises an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators) with which the attachment molecule 526 is capable of bonding or otherwise forming an attachment. In additional embodiments, the anchor molecule 18 comprises a protein, a peptide, an antibody, an aptamer (RNA and DNA), a nanobody, a darpin, a catalyst, a polymerization initiator, a polymer like PEG, an organic molecule or combinations thereof, any or all of which the attachment molecule 526 may be capable of bonding with or otherwise forming an attachment.

In practice, the concentration, absolute number, and/or placement of attachment molecules 526, may be controlled or adjusted to control the density of viable attachment molecules on the surface 520. In this manner a specified number of supramolecular structures 40 may be bound to the surface 520 at an optimal or otherwise desired density. As discussed in greater detail below, in certain implementations this may be combined with precise or targeted placement on the surface 520 for additional utility.

In the depicted example, the sample-facing surface 520 may also comprise or otherwise include one or more additional types of universal adaptor structures 530, which as used herein may be understood to include (but are not limited to) primers that facilitate capture and amplification operations (e.g., ILLUMINA® adaptors, THERMO FISHER® adaptors, and so forth). Such adaptor structures 530 may, during operation, interact with the supramolecular structures 40, such as with an analyte bound to the supramolecular structure 40. By way of example, the adaptor structures 530A and 530B may take the form of one or more types of amplification reagents (e.g., polymerization initiators, such as primers) capable of initiating polymerization in response to individual analyte capture events (i.e., linkage between a capture molecule 2, detector molecule 1, and analyte molecule 44). Polymerization in response to interaction between an amplification reagent and analyte binding event may result in a signaling element 414 being linked proximate to the respective analyte (e.g., linked with the capture and detector molecules).

In some embodiments, the signaling element 414 may be optically active (e.g., fluorescent) and can be measured and/or localized using a microscope or optical sensor provided external to or as part of the substrate. In other embodiments, the signaling element is electrically active and may be measured and/or localized using an electrical sensor provided as part of or external to the substrate. In further embodiments, the signaling element is magnetically active and may be measured and/or localized using a magnetic sensor provided as part of or external to the substrate. In certain implementations, each signal event is associated with the capture of the same type of analyte molecule (a single copy of the same type of analyte molecule), determined by the corresponding detector and capture molecule, thus counting the number of locations where the signaling element is present gives the quantification of the analyte molecule in the sample.

As may be understood with respect to the following examples, and as discussed throughout, in certain implementations a supramolecular structure 40 may be functionalized with affinity binders (e.g., a detector molecule 1 and a capture molecule 2), where the affinity binders may be implemented as antibodies, aptamers, nanobodies, or other suitable affinity binders as discussed herein. Correspondingly, there may be one or two unique identifiers on each supramolecular structure 40 associated with the respective affinity binders, as discussed herein. For example, when one unique identifier is used, it may be the same barcode sequence for both antibodies (in an antibody-based implementation or example) and the two antibodies could be the same or different. In an implementation in which a single affinity binder is employed (e.g., only a capture molecule 2 or a detector molecule 1), there would be only one unique identifier. In the instance where there are three or more affinity binders (e.g., additional capture or detector molecules), there could be one, two, three or more unique identifiers or it could be a single identifier used to define the set of affinity binders on a specific supramolecular structure or combination of supramolecular structures.

As used herein, and discussed throughout, a unique identifier (e.g., capture bridge 7 or detector bridge 8 of FIG. 1) may be an oligomer (e.g., oligonucleotide), a polymer, or it could be a complex combination of unique oligomers that are conjugated to each other. By way of example, one possible complex unique identifier that may be comprised of a combination of oligomers is one that is ready to be integrated into an amplification scheme supported by DNA sequencing.

In such an example, the complex set may be the unique DNA sequence (e.g., barcode 540) associated with the identifier for the affinity binder (e.g., capture molecule 2 or detector molecule 1). This unique DNA sequence may be flanked by (e.g., conjugated to) primers 542A, 542B that are complementary to primers that are grafted into the hydrogel matrix on the surface 520 of the substrate (e.g., P5 and P7, so P5′ and P7′), as shown in FIG. 25. In one such example the adaptor structures 530A and 530B present on the surface 520 of the substrate may be complements to primers 542A, 542B (e.g., complementary primers). Thus, as used herein, the conjugated series 544 of primers 542 and barcode sequence 540 comprises a unique identifier (e.g., capture bridge 7 or detector bridge 8 of FIG. 1) that may be conjugated to an affinity binder (e.g., capture molecule 2 or detector molecule 1) and integrated with a supramolecular structure 40 as described herein.

With the preceding structure of FIG. 25 in mind, when an assay is performed and an analyte of interest is captured by the affinity binders, a subsequent cleaving (e.g., deconstructing) step may be performed that severs the linkage of one of the affinity binders plus the associated unique identifier from the supramolecular structure 40. In this manner, one of the primers (e.g., primer 542A) remains attached to the supramolecular structure 40 on one side while the other primer (e.g., primer 542B) is unattached (i.e., free) in the solution.

This allows for various possible options. By way of example, in accordance with one possible option, the unattached (i.e., free) primer 452B can be captured by the complementary primer 530B via hybridization onto the surface 520 of the substrate. This would then be a second attachment point (e.g., binding) of the supramolecular structure 40 to the surface 520 via hybridization of the primer 542B to the surface 520 (the first being via the anchor molecule 118 and attachment molecule 526 which initially linked the supramolecular structure 40 to the surface 520).

Subsequent to this second, linking hybridization, the linker between the other primer 542A and the supramolecular structure 40 can be cleaved and the conjugated series 544 (including a library element in the form of barcode sequence 540) is bound to the surface 520 via the linkage formed by primer 542B and complementary adaptor 530B. This library element may then be either read out directly or amplified (such as via bridge amplification) to form a cluster and the cluster read out via sequencing.

Alternatively, a separate option is to also sever the linkage between the primer 542A from the supramolecular structure 40, leaving the library element (i.e., barcode sequence 540) free in solution. Once in solution, the library element may be recaptured onto the surface 520 via hybridization between primer 542A and complementary adaptor 530A or primer 542B and complementary adaptor 530B. Once captured to the surface 520, the library element may be read out directly or amplified to form a cluster, which may be read out via sequencing.

### Example 1

With the preceding structures and processing options in mind, various illustrative, but non-limiting, examples are provided to demonstrate contemplated scope of the present techniques as well as to facilitate explanation. By way of example, and turning to FIG. 26, in this example one or more configurations or types of supramolecular structures 40 are bonded to corresponding attachment structures 526 provided on a surface 520 as described above (i.e., using a suitable binding and placement methodology), as shown at Step 550. As previously noted, such binding may be random over the surface 520 or may be at patterned or otherwise ordered locations. As shown in FIG. 26, two sets of additional steps may be performed, an assay operation and a mapping operation. For the purpose of explanation, these steps are shown in parallel (i.e., mapping may be performed independent of (e.g., prior to and by a different entity or person) an assay operation), though they may also be performed sequentially (i.e., a map is generated as a preceding step to an assay being performed by an entity or person who is also performing the assay). In practice the mapping step may be performed by the same entity performing the assay, though it may also, in the alternative, be performed by a different entity, such as by an entity preparing and providing substrates for subsequent use in performing assays. Thus, it should be appreciated that the depicted steps may be performed in series or in parallel and certain of the steps may be performed by different entities.

With respect to the mapping operation, and turning back to FIG. 26, once the surface 520 is seeded with the desired supramolecular structure(s) 40, the barcode sequences 540 associated with affinity binders of the supramolecular structures 40 may be read to generate (Step 562) a decode mapping file 564 that identifies physical, spatial locations on the surface 520 to which the respective supramolecular structures are bound. That is, the decode mapping file 564 associates or maps respective spatial locations on the surface 520 to identified barcode sequences 540, which correspond to library elements.

The mapped surface 520 may then be exposed (Step 554) to one or more samples 552 containing one or more analytes to be detected and/or quantified. By way of example a fluid, analyte-containing sample 552 may be flowed continuously, periodically, or intermittently over the surface 520, such as at a predefined rate, periodicity, volume, and so forth, to bind analyte present in the sample(s) 552 to the affinity binders of the supramolecular structures 40 attached to the surface 520. In certain implementations, an incubation or waiting period 556 (e.g., 10 seconds, 30 seconds, 1 minute, 5 minutes, 10 minutes, and so forth) may be provided as well to facilitate or optimize binding events for subsequent detection. Once the sample 552 has been processed in this manner, the supramolecular structures 40 to which analyte has been bound may be interrogated (Step 558) to identify capture sites 560 corresponding to sites where a supramolecular structure 40 that has captured an analyte molecule is bound to the surface 520, as described herein. By way of example, a deconstruction or cleavage operation as described herein may be performed to expose library elements of affinity binders which have bound an analyte. An amplification operation may be performed for the respective exposed library elements, with one or more fluorescent signaling elements being attached as a result of the amplification steps. By way of example, in one implementation a detector barcode may be used as a polymerization initiator for growth of a highly fluorescent polymer in an amplification process, such as rolling circle amplification or hybridization chain reaction.

The locations of fluorescent labels may then be determined relative to the geometry of the surface 520 (such as using one or more fiducial markers) to generate the capture site data. The capture sites 560 identified in this manner or by a comparable technique may be used in combination with the decode mapping file 564 (e.g., comparison Step 566) to generate quantitative and/or qualitative measures or assessments of analyte capture (Data 570). As may be appreciated, in accordance with this example, read out of the assay results occurs without performing a sequence operation (i.e., without sequencing the respective barcodes corresponding to the library elements). Instead, the spatial location of a binding event relative to the surface 520 is used to identify the respective library element.

While a decode map-based approach is described in this example, it may be appreciated that comparable mechanisms may be employed to achieve spatial or location-based readout without amplification. For example, instead of allowing random distribution of supramolecular structures 40 and subsequently generating a decode map, the supramolecular structures 40 may instead be seeded on the surface 520 in a known or ordered manner (such as by selective or targeted placement of attachment molecules, use of topographical and/or chemical features during placement (e.g., nanowells) and so forth such that locations of given supramolecular structures 40 (and their corresponding affinity binders and library elements) is known. In such a scenario, readout may be accomplished in a comparable manner (e.g., amplification and attachment of a fluorescent signaling element) and generation of results may be performed using the identified spatial capture sites and the known placement of affinity binders and library elements. In such a scenario, the decode map is essentially known based on the targeted or constrained seeding operation.

It should also be appreciated that the work flow shown in FIG. 26 may be modified to take into account and/or leverage variations on the supramolecular structure 40 as discussed herein. By way of example, as described herein the core structure 13 of the supramolecular structure 40 may have attached multiple copies of a barcode or barcodes that are not part of the linkage structures used to link to the affinity binder molecules but which may be used to identify the affinity binders present on the supramolecular structure 40. That is, the supramolecular structure 40 may include multiple copies of one or more barcodes that may be used to identify the affinity binders present on the supramolecular structure 40 and which are not part of the linkage structure used to attach the affinity binders to the supramolecular structure 40.

In such an example, if the seeding of the supramolecular structure 40 with the affinity binders and barcodes occurred during manufacture of the substrate having the surface 520, the decode mapping file 564 may be generated at the time of manufacture to facilitate generation of capture data subsequently. The decode mapping data 564 for the substrate may be generated with or without amplification of the copies of the barcode(s) seeded on the supramolecular structure 40. In particular, depending on the readout mechanism, the multiple copies of the barcode present on the core structure 13 may allow the decode mapping file 564 to be generated without amplification due to the increased signal associated with the use of multiple barcodes for each supramolecular structure 40. Even in such a context, some amount of amplification may also be performed if it is determined to be beneficial. In this scenario where the decode mapping file 564 is generated beforehand, the workflow of the end user may be sped up due to the user not having to perform the decode mapping operation. Instead, in one embodiment the user would simply amplify a probe (e.g., oligomer) and look for a signal and then compare (step 566) the location (i.e., capture sites 560) of where the signal is observed with the decode mapping file 564.

In workflows in which the user seeds the core structure 13 to form the supramolecular structure 40, decoding of the placement of the supramolecular structures 40 may be performed as described above, either by sequencing or decoding the barcodes through the use of a deconstruction or cleavage operation. Use of the resulting decode mapping file 564 may otherwise proceed as described above.

### Example 2

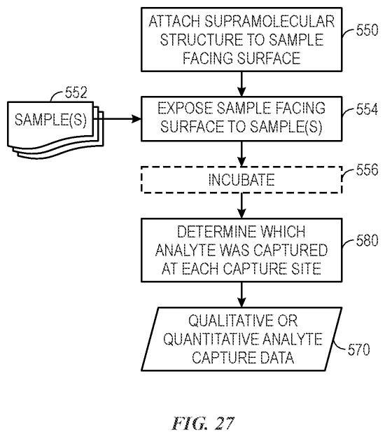

Turning to FIG. 27, a further example is provided. In this example readout is not based on spatial locations (e.g., a decode map or nanowell placement). As shown in FIG. 27, initial steps correspond generally to steps described with respect to FIG. 26. For example, one or more configurations or types of supramolecular structures 40 are bonded to corresponding attachment structures 526 provided on a surface 520 as described above (i.e., using a suitable binding and placement methodology), as shown at Step 550. Such binding may be random over the surface 520 or may be at patterned or otherwise ordered locations. The seeded surface 520 may then be exposed (Step 554) to one or more samples 552 containing one or more analytes to be detected and/or quantified. By way of example a fluid, analyte-containing sample 552 may be flowed continuously, periodically, or intermittently over the surface 520, such as at a predefined rate, periodicity, volume, and so forth, to bind analyte present in the sample(s) 552 to the affinity binders of the supramolecular structures 40 attached to the surface 520. In certain implementations, an incubation or waiting period 556 (e.g., 10 seconds, 30 seconds, 1 minute, 5 minutes, 10 minutes, and so forth) may be provided as well to facilitate or optimize binding events for subsequent detection. Once the sample 552 has been processed in this manner, the supramolecular structures 40 to which analyte has been bound may be interrogated (Step 580) to identify which sites having a supramolecular structure 40 have captured an analyte molecule and which analyte(s) (in a multi-analyte assay) have been captured. In practice this may be accomplished by reading the barcodes of the affinity binders at the identified capture sites, as discussed herein. For example, in certain embodiments, sequence-based approaches may be employed to read-out the barcodes associated with an analyte capture event. In other embodiments, amplification approaches may be employed, with different characteristics signaling elements attached to different barcodes, thereby allowing the use of imaging-based approaches to identify and distinguish capture events associated with different analytes. In this manner, quantitative and/or qualitative measures or assessments of analyte capture (Data 570) maybe generated without spatial decode data.

### Example 3

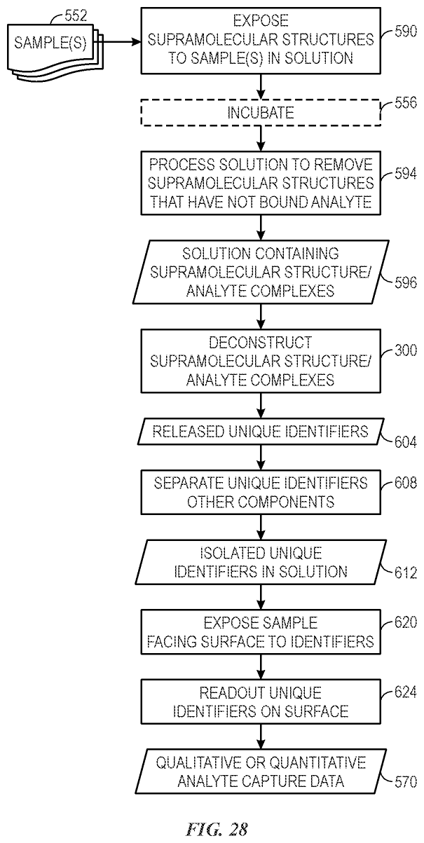

Turning to FIG. 28, a further example is provided. In this example, analyte binding occurs in a solution-phase, as opposed to on a surface 520. As shown in FIG. 28, one or more configurations or types of supramolecular structures 40 are provided in solution. One or more samples 552 containing one or more analytes to be detected and/or quantified may be combined in solution (Step 590) with the supramolecular structures 40. By way of example a fluid, analyte-containing sample 552 may be combined with a solution containing supramolecular structures 40, or vice versa. In certain implementations, an incubation or waiting period 556 (e.g., 10 seconds, 30 seconds, 1 minute, 5 minutes, 10 minutes, and so forth) may be provided as well to facilitate or optimize binding events for subsequent detection.

The solution containing the supramolecular structures 40 and analyte may be processed (Step 594) to separate those supramolecular structures that have captured the analyte of interest from those that have not. The resulting solution 596, containing supramolecular structures that have captured analyte molecules may be exposed or have added a deconstructor molecule (Step 600), which as described herein may act to cleave the respective unique identifier(s) (e.g., library elements or barcodes) from the supramolecular structures 40 in solution to yield released unique identifiers 604. The released unique identifiers 604 may then be separated (Step 608) from the remaining solution to derive isolated unique identifiers 612. As may be appreciated, in certain implementations release of the unique identifiers may correspond to creation of a DNA library such that the analyte capture event corresponds to a DNA library creation event. In certain contexts this effectively transforms a protein capture assay into a DNA sequencing library preparation kit.

Turning back to FIG. 28, the isolated unique identifiers 612 may then be flowed over a prepared surface 520 (Step 620) as discussed herein and bound to corresponding (e.g., complementary) adapters seeded on the surface 520. The unique identifiers attached to the surface 520 may then be read-out (step 624) as discussed herein (e.g., via an amplification to attach a signaling element, a sequencing operation, and so forth, and resulting qualitative or quantitative analyte capture data 570 generated. As previously described, the surface may be randomly seeded with adaptors or may be seeded in accordance with an order or pattern and generation of the analyte capture data 570 may, as appropriate, utilize decode mapping data. This approach provides certain advantages, including resolving a three-dimensional (3d) capture problem when in solution to a 2D capture problem when capture is performed on the surface 520.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for generating analyte capture data, the method comprising:
attaching a plurality of supramolecular structures to a surface of a substrate, wherein the supramolecular structures each comprise one or more affinity binders attached to a core structure;
exposing the surface to a sample comprising an analyte;
interrogating the surface to identify one or more capture sites of bound analytes; and
comparing the capture sites to a decode mapping file to generate qualitative or quantitative analyte capture data.

2. The method of claim 1, wherein the core structure comprises a DNA origami.

3. The method of claim 1, wherein the substrate comprises a flow cell or a portion of a flow-cell.

4. The method of claim 1, wherein the affinity binders comprise one or more of a protein, a peptide, an antibody, an aptamer, a fluorophore, a darpin, a catalyst, a polymerization initiator, a polymer, or combinations thereof.

5. The method of claim 1, further comprising incubating the surface and the sample.

6. The method of claim 1, wherein the decode mapping file comprises location data relating placement of supramolecular structures and associated affinity binders on the surface.

7. A method for generating analyte capture data, the method comprising:
attaching a plurality of supramolecular structures to a surface of a substrate, wherein the supramolecular structures each comprise one or more affinity binders attached to a core structure;
generating a decode mapping file for the surface, wherein decode mapping file comprises location data relating placement of supramolecular structures and associated affinity binders on the surface; and
comparing one or more capture sites of one or more analytes on the surface after exposure to a sample to the decode mapping file to generate qualitative or quantitative analyte capture data.

8. The method of claim 7, wherein the core structure comprises a DNA origami.

9. The method of claim 7, wherein the substrate comprises a flow cell or a portion of a flow-cell.

10. The method of claim 7, wherein the affinity binders comprise one or more of a protein, a peptide, an antibody, an aptamer, a fluorophore, a darpin, a catalyst, a polymerization initiator, a polymer, or combinations thereof.

11. A method for generating analyte capture data, the method comprising:
attaching a plurality of supramolecular structures to a surface of a substrate, wherein the supramolecular structures each comprise one or more affinity binders attached to a core structure;
exposing the surface to a sample comprising one or more analytes; and
determining which analyte was captured at each capture site on the surface to generate qualitative or quantitative analyte capture data.

12. The method of claim 11, wherein the core structure comprises a DNA origami.

13. The method of claim 11, wherein the substrate comprises a flow cell or a portion of a flow-cell.

14. The method of claim 11, wherein the affinity binders comprise one or more of a protein, a peptide, an antibody, an aptamer, a fluorophore, a darpin, a catalyst, a polymerization initiator, a polymer, or combinations thereof.

15. The method of claim 11, further comprising incubating the surface and the sample.

16. A method for generating analyte capture data, the method comprising:
exposing, in a solution, a plurality of supramolecular structures to a sample comprising one or more analytes, wherein the supramolecular structures each comprise one or more affinity binders attached to a core structure;
processing the solution to remove supramolecular structures that have not bound analyte;
deconstructing complexes of the supramolecular structure and analyte present in the solution to release one or more unique identifiers associated with each binding event of analyte to a respective supramolecular structure;
isolating the one or more unique identifiers in the solution;
exposing a surface seeded with a one or more adapter types to the solution comprising the one or more unique identifiers; and
performing a readout operation of the one or more unique identifiers which have interacted with the one or more adapters to generate qualitative or quantitative analyte capture data.

17. The method of claim 16, further comprising incubating the supramolecular structures and the sample in solution.

18. The method of claim 16, wherein the one or more unique identifiers comprise barcode sequences.

19. The method of claim 16, wherein the one or more adapter types comprise one or more primers, wherein at least a subset of the one or more primers are complementary to at least a portion of the one or more unique identifiers.

20. The method of claim 16, wherein the core structure comprises a DNA origami.

21. The method of claim 16, wherein the surface is provided as a portion of a flow cell.

22. The method of claim 16, wherein the affinity binders comprise one or more of a protein, a peptide, an antibody, an aptamer, a fluorophore, a darpin, a catalyst, a polymerization initiator, a polymer, or combinations thereof.

23. A method for generating analyte capture data, the method comprising:
exposing, in a solution, a plurality of supramolecular structures to a sample comprising one or more analytes, wherein the supramolecular structures each comprise one or more affinity binders attached to a core structure;
processing the solution to remove supramolecular structures that have bound analyte;
deconstructing supramolecular structures unbound to analytes and remaining in the solution to release one or more unique identifiers indicative of which analyte or analytes to which the remaining supramolecular structures have an affinity;
isolating the one or more unique identifiers in the solution;
exposing a surface seeded with a one or more adapter types to the solution comprising the one or more unique identifiers; and
performing a readout operation of the one or more unique identifiers which have interacted with the one or more adapters to generate qualitative or quantitative analyte capture data.

24. A substrate comprising a plurality of supramolecular structures, each supramolecular structure comprising:
a core structure comprising a plurality of core molecules;
one or more capture molecules linked to the supramolecular core at a first set of locations;
one or more detector molecules linked to the supramolecular core at a second set of locations; and
one or more barcode sequences linked to the supramolecular core at locations other than the first set of locations and the second set of locations, wherein the one or more barcode sequences identify the core structure, the capture molecules, the detector molecules, or combinations thereof;
wherein one or both of the capture molecules or the detector molecules selectively bind to an analyte of interest.

25. The substrate of claim 24, wherein the barcode sequences comprise nucleic acid sequences.

26. The substrate of claim 24, wherein each core structure of the plurality of supramolecular structures is identical to each other.

27. The substrate of claim 24, wherein the substrate comprises a solid support, solid substrate, a polymer matrix, or a molecular condensate.

28. The substrate of claim 24, wherein the analyte of interest comprises a protein, a peptide, a peptide fragment, a lipid, a DNA, a RNA, an organic molecule, an inorganic molecule, complexes thereof, or any combinations thereof.

29. The substrate of claim 24, wherein each supramolecular structure is a nanostructure.

30. The substrate of claim 24, wherein the plurality of core molecules for each core structure comprises one or more nucleic acid strands, one or more branched nucleic acids, one or more peptides, one or more small molecules, or combinations thereof.

31. The substrate of claim 24, wherein each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

32. The substrate of claim 24, wherein the one or more capture molecules and one or more detector molecules for each supramolecular structure independently comprise a protein, a peptide, an antibody, an aptamer (RNA and DNA), a fluorophore, a darpin, a catalyst, a polymerization initiator, a polymer, or combinations thereof.
